(19) United States
(12) Patent Application Publication
(10) Pub. No.: US 2005/0039271 A1
(43) Pub. Date: Feb. 24, 2005

(54) Colouring agents

(76) Inventors: Erik Schulze zur Wiesche, Hamburg (DE); Detlef Hollenberg, Erkrath (DE); Horst Hoeffkes, Duesseldorf (DE); Britta Bossmann, Erkrath (DE)

Correspondence Address: HENKEL CORPORATION, THE TRIAD, SUITE 200, 2200 RENAISSANCE BLVD., GULPH MILLS, PA, 19406, US

(21) Appl. No.: 10/929,025

(22) Filed: Aug. 27, 2004

Related U.S. Application Data
(63) Continuation of application No. PCT/EP03/01648, filed on Feb. 19, 2003.

(30) Foreign Application Priority Data
Mar. 1, 2002 (DE) 102 08 874.8
Jul. 6, 2004 (DE) 102 30 415.7

(51) Int. Cl. A61K 7/13

(52) U.S. Cl. 8/405

(57) ABSTRACT

A shaped body for coloring keratinous fibers consisting of at least one dissolution accelerator and at least one oxidation dye precursor of the secondary intermediate type, contained within a cosmetically acceptable carrier. The shaped body is free from oxidation dye precursors of the primary intermediate type. Also disclosed are a method for coloring keratin fibers and a kit containing these ingredients.

### CROSS REFERENCE TO RELATED APPLICATIONS

This application is a continuation under 35 U.S.C. § 365(c) and 35 U.S.C. § 120 of international application PCT/EP03/01648, filed on Feb. 19, 2003. This application also claims priority under 35 U.S.C. § 119 of DE 102 08 874.8, filed Mar. 1, 2002 and DE 102 30 415.7, filed Jul. 6, 2002, each of which is incorporated herein by reference in its entirety.

### BACKGROUND OF THE INVENTION

This invention relates to shaped bodies for coloring keratinous fibers which contain at least one oxidation dye precursor of the secondary intermediate type and which are free from oxidation dye precursors of the primary intermediate type, to the use of these compositions for the production of hair coloring preparations, to a process for coloring keratinous fibers using these shaped bodies and to a kit for use in this process.

Nowadays, human hair is treated in many different ways with hair-care preparations. Such treatments include, for example, the cleaning of hair with shampoos, the care and regeneration of hair with rinses and treatments and the bleaching, coloring and shaping of hair with coloring and tinting formulations, wave formulations and styling preparations. Among these, formulations for modifying or shading the color of the hair occupy a prominent position.

Colorants or tints containing substantive dyes as their coloring component are normally used for temporary colors. Substantive dyes are based on dye molecules which are directly absorbed onto the hair and do not require an oxidative process for developing the color. Dyes such as these include, for example, henna which has been used since ancient times for coloring the body and hair. Corresponding colors are generally much more sensitive to shampooing than oxidative colors so that an often unwanted change of shade or even a visible “decoloration” can occur very much more quickly.

So-called oxidation colorants are used for permanent, intensive colors with corresponding fastness properties. Oxidation colorants normally contain oxidation dye precursors, so-called primary intermediates and secondary intermediates. The primary intermediates form the actual dyes with one another or by coupling with one or more secondary intermediates under the influence of oxidizing agents or atmospheric oxygen. Combinations of oxidation dyes and substantive dyes are often also used to obtain special shades. Oxidation colorants are distinguished by excellent long-lasting coloring results. Natural-looking colors normally require a mixture of a relatively large number of oxidation dye precursors; in many cases, substantive dyes are used for shading.

Finally, a new coloring process has recently attracted considerable interest. In this process, precursors of the natural hair dye melanin are applied to the hair and, in the course of oxidative processes, form “nature-analogous” dyes in the hair. One such process using 5,6-dihydroxyindoline as a dye precursor was described in EP-B1 530 229. By applying preparations containing 5,6-dihydroxyindoline, in particular repeatedly, people with gray hair can be given back their natural hair color. Development can be carried out with atmospheric oxygen as the sole oxidizing agent, so that there is no need to use other oxidizing agents. With people originally having medium-blond to brown hair, indoline may be used as the sole dye precursor. By contrast, for use in people originally having red and, in particular, dark or black hair, satisfactory results can often only be achieved by the additional use of other dye components, more particularly special oxidation dye precursors.

Hair colorants are normally formulated as aqueous emulsions or gels which are optionally mixed with an oxidizing preparation immediately before application. However, this process is unsatisfactory in regard to the storage stability of the formulations, their dosability and their ease of handling.

Another possibility is to formulate hair colorants as solids in the form of powders or tablets. Hair colorants of this type are usually dissolved in water while stirring immediately before application. The resulting ready-to-use colorant is generally a gel or cream and is then applied to the hair. Where the colorant is formulated as a solid, its dissolving behavior is critical. The solid should not form lumps because this impairs the effectiveness of the ready-to-use colorant. Besides the optimal Theological properties of the colorant, rapid dissolving of the solid is desirable, particularly where the colorant is formulated as a tablet of whatever form.

DE-A-36 09 962 discloses a tablet-form colorant based on henna and oxidation dye precursors which is said to give intensive black colors after only very short contact times. However, there is no reference whatever in this document to the shaped bodies according to the invention for coloring hair.

DE-A1-199 61 910 discloses shaped bodies for coloring keratin fibers which, as multiphase tablets, have to contain at least one dye precursor in one phase and an oxidizing agent in another phase. The tablets are dissolved in water in a corresponding coloring process.

WO 01/45655 discloses shaped bodies for coloring keratin fibers which contain indole or indoline derivatives as oxidation dye precursors of the primary intermediate type. These shaped bodies are used in a process for coloring keratin fibers. To produce the ready-to-use colorant, the shaped body is dissolved in water.

WO 01/45654 discloses colorants in the form of a shaped body which contains at least one synthetic substantive dye. These shaped bodies are used in a process for coloring keratin fibers in which the shaped body is dissolved in water to produce the ready-to-use colorant.

With all the shaped bodies mentioned above, both dissolving behavior, particularly in viscous media, such as creams for example, and the rheology of the mixture applied are in need of improvement. In addition, the stability of the components in the known shaped bodies, above all against oxidative influences, is unsatisfactory.

Accordingly, the problem addressed by the present invention was to improve the shaped bodies in regard to their dissolving behavior and the mixture applied in regard to its rheology and, at the same time, to obtain optimal coloring properties.

### SUMMARY OF THE INVENTION

It has now surprisingly been found that, by using the shaped bodies according to the invention, the colors obtained can be distinctly improved in regard to their intensity and fastness properties and that the shaped bodies are distinguished by a distinctly reduced dissolving time.

### DETAILED DESCRIPTION OF THE INVENTION

In a first embodiment, therefore, the present invention relates to shaped bodies for coloring keratinous fibers which, besides a cosmetically acceptable carrier, contain at least one dissolution accelerator and at least one oxidation dye precursor of the secondary intermediate type and which are free from oxidation dye precursors of the primary intermediate type.

Keratinous fibers in the context of the invention are understood to be pelts, wool, feathers and, in particular, human hair.

m-Phenylenediamine derivatives, naphthols, resorcinol and resorcinol derivatives, pyrazolones and m-aminophenol derivatives are generally used as oxidation dye precursors of the secondary intermediate type. Particularly suitable secondary intermediates are 1-naphthol, 1,5-, 2,7- and 1,7-dihydroxynaphthalene, 5-amino-2-methylphenol, m-aminophenol, resorcinol, resorcinol monomethyl ether, m-phenylenediamine, 1-phenyl-3-methyl-5-pyrazolone, 2,4-dichloro-3-aminophenol, 1,3-bis-(2′,4′-diaminophenoxy)-propane, 2-chlororesorcinol, 4-chlororesorcinol, 2-chloro-6-methyl-3-aminophenol, 2-amino-3-hydroxypyridine, 2-methyl resorcinol, 5-methyl resorcinol and 2-methyl-4-chloro-5-aminophenol.

According to the invention, preferred secondary intermediates are
m-aminophenol and derivatives thereof such as, for example, 5-amino-2-methylphenol, N-cyclopentyl-3-aminophenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2′-hydroxyethyl)-amino-2-methylphenol, 3-(diethylamino)-phenol, N-cyclopentyl-3-aminophenol, 1,3-dihydroxy-5-(methylamino)-benzene, 3-(ethylamino)-4-methylphenol and 2,4-dichloro-3-aminophenol,
o-aminophenol and derivatives thereof,
m-diaminobenzene and derivatives thereof such as, for example, 2,4-diaminophenoxyethanol, 1,3-bis-(2′,4′-diaminophenoxy)-propane, 1-methoxy-2-amino-4-(2′-hydroxyethylamino)-benzene, 1,3-bis-(2′,4′-diaminophenyl)-propane, 2,6-bis-(2′-hydroxyethylamino)-1-methyl-benzene and 1-amino-3-bis-(2′-hydroxyethyl)-aminobenzene,
o-diaminobenzene and derivatives thereof such as, for example, 3,4-diaminobenzoic acid and 2,3-diamino-1-methylbenzene,
di- and trihydroxybenzene derivatives such as, for example, resorcinol, resorcinol monomethyl ether, 2-methyl resorcinol, 5-methyl resorcinol, 2,5-dimethyl resorcinol, 2-chlororesorcinol, 4-chlororesorcinol, pyrogallol and 1,2,4-trihydroxybenzene,
pyridine derivatives such as, for example, 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine and 3,5-diamino-2,6-dimethoxypyridine,
naphthalene derivatives such as, for example, 1-naphthol, 2-methyl-1-naphthol, 2-hydroxymethyl-1-naphthol, 2-hydroxyethyl-1-naphthol, 1,5-dihydroxynaphthalene, 1,6-dihdroxynaphthalene, 1,7-dihdroxy-naphthalene, 1,8-dihdroxynaphthalene, 2,7-dihdroxynaphthalene and 2,3-dihdroxynaphthalene,
morpholine derivatives such as, for example, 6-hydroxybenzomorpholine and 6-aminobenzomorpholine,
quinoxaline derivatives such as, for example, 6-methyl-1,2,3,4-tetrahydroquinoxaline, pyrazole derivatives such as, for example, 1-phenyl-3-methylpyrazol-5-one,
indole derivatives such as, for example, 4-hydroxyindole, 6-hydroxyindole and 7-hydroxyindole,
pyrimidine derivatives such as, for example, 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine and 4,6-dihydroxy-2-methylpyrimidine or
methylenedioxybenzene derivatives such as, for example, 1-hydroxy-3,4-methylenedioxybenzene, 1-amino-3,4-methylenedioxybenzene and 1-(2′-hydroxyethyl)-amino-3,4-methylenedioxybenzene.

In a particularly preferred embodiment, the shaped body according to the invention contains at least one oxidation dye precursor of the secondary intermediate type selected from 1-naphthol, 1,5-, 2,7- and 1,7-dihydroxynaphthalene, 3-aminophenol, 5-amino-2-methylphenol, 2-amino-3-hydroxypyridine, 3-amino-2-methylamino-6-methoxypyridine, resorcinol, 4-chlororesorcinol, 2,4-diaminophenoxyethanol, 2-chloro-6-methyl-3-aminophenol, 2-methyl resorcinol, 5-methyl resorcinol, 2,5-dimethyl resorcinol and 2,6-dihydroxy-3,4-dimethylpyridine.

So far as the secondary intermediates suitable for use in the shaped bodies according to the invention are concerned, reference is also expressly made to to the work by Ch. Zviak, The Science of Hair Care, Chapter 7 (pages 248-250; substantive dyes) and Chapter 8, pages 264-267; oxidation dye precursors), published as Volume 7 of the Series “Dermatology” (Ed.: Ch. Culnan and H. Maibach), Marcel Dekker Inc., New York/Basle, 1986, and to the “Europäische Inventar der Kosmetik-Rohstoffe” published by the Europäische Gemeinschaft and available in disk form from the Bundesverband Deutscher Industrie- und Handelsunternehmen fur Arzneimittel, Reformwaren und Körperpflegemittel d.V., Mannheim, Germany.

The shaped body according to the invention contains at least one dissolution accelerator. The term “dissolution accelerator” encompasses gas-evolving components, preformed and enclosed gases, disintegration aids and mixtures thereof.

In a first embodiment of the present invention, gas-evolving components are used as the dissolution accelerator. Such components react with one another on contact with water to form gases in situ which generate a pressure in the tablet that causes the tablet to disintegrate into relatively small particles. One example of such a system are special combinations of suitable acids with bases. Mono-, di- or tribasic acids with a pKₐ value of 1.0 to 6.9 are preferred. Preferred acids are citric acid, malic acid, maleic acid, malonic acid, itaconic acid, tartaric acid, oxalic acid, glutaric acid, glutamic acid, lactic acid, fumaric acid, glycolic acid and mixtures thereof. Citric acid is particularly preferred. In a particularly preferred embodiment, the citric acid is used in particle form, the particles having a diameter below 1,000 μm, preferably below 700 μm and more particularly below 400 μm. Other alternative suitable acids are the homopolymers or copolymers of acrylic acid, maleic acid, methacrylic acid or itaconic acid with a molecular weight of 2,000 to 200,000. Homopolymers of acrylic acid and copolymers of acrylic acid and maleic acid are particularly preferred. According to the invention, preferred bases are alkali metal silicates, carbonates, hydrogen carbonates and mixtures thereof. Metasilicates, hydrogen carbonates and carbonates are particularly preferred, hydrogen carbonates being most particularly preferred. Particulate hydrogen carbonates with a particle diameter below 1,000 μm, preferably below 700 μm and more particularly below 400 μm are particularly preferred. Sodium or potassium salts of the bases mentioned above are particularly preferred. The gas-evolving components are present in the shaped bodies according to the invention in a quantity of preferably at least 10% by weight and more particularly at least 20% by weight.

In a second embodiment of the present invention, the gas is preformed or enclosed so that the evolution of gas begins as the shaped body begins to dissolve and accelerates the dissolving process. Examples of suitable gases are air, carbon dioxide, N₂O, oxygen and/or other non-toxic, non-inflammable gases.

In a particularly preferred embodiment of the present invention, disintegration aids, so-called tablet disintegrators, are incorporated in the shaped bodies to shorten their disintegration times. According to Römpp (9th Edition, Vol. 6, page 4440) and Voigt “Lehrbuch der pharmazeutischen Technologie” (6th Edition, 1987, pages 182-184), tablet disintegrators or disintegration accelerators are auxiliaries which promote the rapid disintegration of tablets in water or gastric juices and the release of the pharmaceuticals in an absorbable form.

These substances, which are also known as “disintegrators” by virtue of their effect, undergo an increase in volume on contact with water (swelling). Swelling disintegration aids are, for example, synthetic polymers, such as polyvinyl pyrrolidone (PVP), or natural polymers and modified natural substances, such as cellulose and starch and derivatives thereof, alginates or casein derivatives.

According to the invention, preferred disintegrators are cellulose-based disintegrators, so that preferred shaped bodies contain a cellulose-based disintegrator in quantities of 0.5 to 70% by weight and preferably 3 to 30% by weight, based on the shaped body as a whole. Pure cellulose has the formal empirical composition (C₆H₁₀O₅)ₙ and, formally, is a β-1,4-polyacetal of cellobiose which, in turn, is made up of two molecules of glucose. Suitable celluloses consist of ca. 500 to 5000 glucose units and, accordingly, have average molecular weights of 50,000 to 500,000. According to the invention, cellulose derivatives obtainable from cellulose by polymer-analog reactions may also be used as cellulose-based disintegrators. These chemically modified celluloses include, for example, products of esterification or etherification reactions in which hydroxy hydrogen atoms have been substituted. However, celluloses in which the hydroxy groups have been replaced by functional groups that are not attached by an oxygen atom may also be used as cellulose derivatives. The group of cellulose derivatives includes, for example, alkali metal celluloses, carboxymethyl cellulose (CMC), cellulose esters and ethers and aminocelluloses. The cellulose derivatives mentioned are preferably not used on their own, but rather in the form of a mixture with cellulose as cellulose-based disintegrators. The content of cellulose derivatives in mixtures such as these is preferably below 50% by weight and more preferably below 20% by weight, based on the cellulose-based disintegrator. In one particularly preferred embodiment, pure cellulose free from cellulose derivatives is used as the cellulose-based disintegrator.

According to the invention, the cellulose used as disintegration aid cannot be used in fine-particle form, but is converted into a coarser form, for example by granulation or compacting, before it is added to and mixed with the premixes to be tabletted. The particle sizes of such disintegration aids is mostly above 200 μm, at least 90% by weight of the particles being between 300 and 1600 μm in size and, more particularly, between 400 and 1200 μm in size. The disintegration aids according to the invention are commercially obtainable, for example under the name of Arbocel® TF-30-HG from Rettenmaier. A preferred disintegration aid is, for example, Arbocel® TF-30-HG.

Microcrystalline cellulose is used as a preferred cellulose-based disintegration aid or as part of such a component. This microcrystalline cellulose is obtained by partial hydrolysis of celluloses under conditions which only attack and completely dissolve the amorphous regions (ca. 30% of the total cellulose mass) of the celluloses, but leave the crystalline regions (ca. 70%) undamaged. Subsequent de-aggregation of the microfine celluloses formed by hydrolysis provides the microcrystalline celluloses which have primary particle sizes of ca. 5 μm and which can be compacted, for example, to granules with a mean particle size of 200 μm. A suitable microcrystalline cellulose is commercially obtainable, for example, under the name of Avicel®.

According to the invention, the accelerated dissolution of the shaped bodies can also be achieved by pregranulation of the other ingredients of the shaped body.

In a preferred embodiment, the shaped bodies according to the invention contain a mixture of starch and at least one saccharide, more particularly in addition to at least one cellulose-based disintegrator, in order to accelerate dissolution. Disaccharides are the preferred saccharides of this embodiment. The ratio by weight of starch to saccharides in the mixture is preferably 10:1 to 1:10, more preferably 1:1 to 1:10 and most preferably 1:4 to 1:8.

The disaccharides used are preferably selected from lactose, maltose, sucrose, trehalose, turanose, gentiobiose, melibiose and cellobiose. Lactose, maltose and sucrose are particularly preferred, lactose being most particularly preferred for the shaped bodies according to the invention.

The starch/saccharide mixture is present in the shaped body in a quantity of 5 to 70% by weight and preferably in a quantity of 20 to 40% by weight, based on the weight of the tablet as a whole.

Although the shaped bodies according to the invention can form mildly acidic, neutral or even alkaline preparations as they dissolve, the shaped bodies according to the invention, in a preferred embodiment, contain at least one alkalizing agent.

In principle, there are no limits to the alkalizing agents. Suitable alkalizing agents are, for example, ammonium salts, carbonates, hydrogen carbonates, phosphates, amino acids, alkali metal or alkaline earth metal hydroxides and organic amines.

A preferred embodiment of the invention is characterized by the use of solid alkalizing agents.

Another preferred embodiment of the invention is characterized by the use of alkalizing agents distinguished by ready solubility in water. In the context of the invention, readily water-soluble compounds are compounds of which at least 5 g dissolves in 100 ml water at 15° C. Compounds with a solubility in water of more than 7.5 g in 100 ml water at 15° C. are particularly preferred.

In a preferred embodiment of the present invention, amino acids or oligopeptides containing at least one amino group and a carboxy or sulfo group, of which a 2.5% aqueous solution has a pH above 9.0, are used as alkalizing agents.

In this embodiment, aminocarboxylic acids—more especially α-aminocarboxylic acids and w-aminocarboxylic acids—are particularly preferred. Of the α-aminocarboxylic acids, lysine and especially arginine are particularly preferred.

The amino acids may be addded to the shaped bodies according to the invention preferably in free form. In a number of cases, however, the amino acids may also be used in salt form. In that case, preferred salts are the compounds with hydrohalic acids, more particularly the hydrochlorides and the hydrobromides.

In addition, the amino acids may also be used in the form of oligopeptides and protein hydrolyzates providing steps are taken to ensure that the necessary quantities of the amino acids used in accordance with the invention are present. In this connection, reference is expressly made to the disclosure of DE-OS 22 15 303.

A most particularly preferred alkalizing agent is arginine, particularly in free form, but also as the hydrochloride, because—apart from its alkaline properties—it also distinctly increases the penetration capacity of the dyes.

The alkalizing agent is present in the shaped bodies according to the invention in quantities of preferably 0.5 to 20% by weight and more particularly 5 to 15% by weight, based on the composition as a whole.

According to the invention, it may be desirable to integrate substantive dyes in the shaped bodies. Nitro dyes have proved to be particularly suitable. In the context of the invention, nitro dyes are understood to be the coloring components which have at least one aromatic ring system that contains at least one nitro group.

Particularly preferred nitro dyes are HC Yellow 2, HC Yellow 4, HC Yellow 5, HC Yellow 6, HC Yellow 12, HC Orange 1, HC Red 1, HC Red 3, HC Red 10, HC Red 11, HC Red 13, HC Red BN, HC Blue 2, HC Blue 12, HC Violet 1 and also 1,4-diamino-2-nitrobenzene, 2-amino4-nitrophenol, 1,4-bis-(β-hydroxyethyl)-amino-2-nitrobenzene, 3-nitro-4-(β-hydroxyethyl)-aminophenol, 2-(2′-hydroxyethyl)-amino-4,6-dinitrophenol, 1-(2′-hydroxyethyl)-amino-4-methyl-2-nitrobenzene, 1-amino-4-(2′-hydroxyethyl)-amino-5-chloro-2-nitrobenzene, 4-amino-3-nitrophenol, 1-(2′-ureidoethyl)-amino-4-nitrobenzene, 4-amino-2-nitrodiphenylamine-2′-carboxylic acid, 6-nitro-1,2,3,4-tetrahydro-quinoxaline, picramic acid and salts thereof, 2-amino-6-chloro-4-nitrophenol, 4-ethylamino-3-nitrobenzoic acid and 2-chloro-6-ethylamino-1-hydroxy-4-nitrobenzene.

Besides nitro dyes, azo dyes, anthraquinones and naphthoquinones are also preferred synthetic substantive dyes for the purposes of the invention. Preferred substantive dyes of this type are, for example, Disperse Orange 3, Disperse Blue 3, Disperse Violet 1, Disperse Violet 4, Acid Violet 43, Disperse Black 9 and Acid Black 52 and also 2-hydroxy-1,4-naphthoquinone.

In another preferred embodiment of the invention, the synthetic substantive dye may contain a cationic group. Particularly preferred are
(i) cationic triphenylmethane dyes,
(ii) aromatic systems substituted by a quaternary nitrogen group and
(iii) substantive dyes containing a heterocycle with at least one quaternary nitrogen.

Examples of class (i) dyes are, in particular, Basic Blue 7, Basic Blue 26, Basic Violet 2 and Basic Violet 14.

Examples of class (ii) dyes are, in particular, Basic Yellow 57, Basic Red 76, Basic Blue 99, Basic Brown 16 and Basic Brown 17.

Examples of class (iii) dyes are disclosed in particular in claims 6 to 11 of EP-A2-998,908 to which reference is explicitly made. Preferred cationic substantive dyes of group (iii) are, in particular, the following compounds:

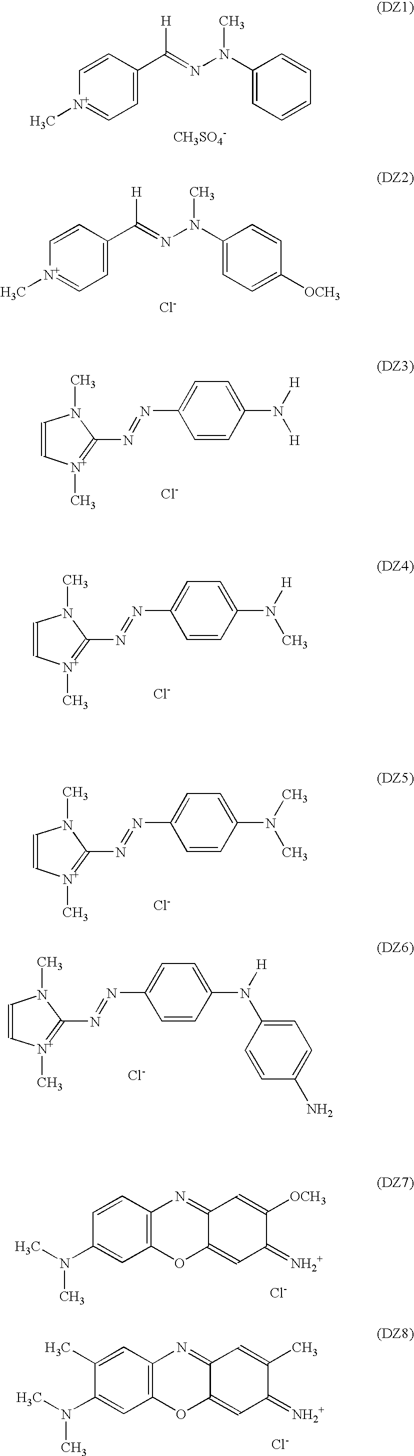

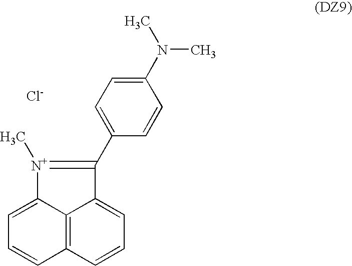

The compounds corresponding to formula (DZ1), (DZ3) and (DZ5) are most particularly preferred cationic substantive dyes of group (iii).

The preparations according to the invention may also contain naturally occurring dyes such as, for example, henna red, henna neutral, henna black, camomile blossom, sandalwood, black tea, black alder bark, sage, logwood, madder root, catechu, sedre and alkanet.

The shaped bodies according to the invention preferably contain the substantive dyes in a quantity of 0.01 to 20% by weight.

In a particularly preferred embodiment, the shaped bodies contain at least one pearlescent pigment. Commonly used pearlescent pigments are natural pearlescent pigments such as, for example, pearl essence (guanine/hypoxanthine mixed crystals from fish scales) or mother-of-pearl (from ground mussel shells), monocrystalline pearlescent pigments, such as bismuth oxychloride for example, and pearlescent pigments based on mica or mica/metal oxide. The last-mentioned pearlescent pigments are provided with a metal oxide coating. Luster and, optionally, color effects are obtained in the shaped bodies according to the invention through the use of the pearlescent pigments. However, the coloring effect of the pearlescent pigments used in the shaped bodies according to the invention does not affect the final result of the coloring of the keratin fibers.

Pearlescent pigments based on mica and on mica/metal oxide are preferred for the purposes of the invention. Mica is one of the layer silicates. The most important representatives of these silicates are muscovite, phlogopite, paragonite, biotite, lepidolite and margarite. To produce the pearlescent pigments in conjunction with metal oxides, the mica—mainly muscovite or phlogopite—is coated with a metal oxide. Suitable metal oxides are inter alia TiO₂, Cr₂O₃ and Fe₂O₃. Interference pigments and bright color pigments are obtained as pearlescent pigments according to the invention by corresponding coating. Besides a glittering optical effect, these types of pearlescent pigments also have color effects. In addition, the pearlescent pigments usable in accordance with the invention may contain a colored pigment which is not based on a metal oxide.

The particle size of the pearlescent pigments preferably used is preferably between 1.0 and 100 μm and more particularly between 5.0 and 60.0 μm.

Particularly preferred pearlescent pigments are the pigments marketed by Merck under the name of Colorona®, the pigments Colorona® red-brown (47-57% by weight muscovite mica (KH₂(AISiO₄)₃), 43-50% by weight Fe₂O₃ (INCl: Iron Oxides Cl 77491), <3% by weight TiO₂ (INCl: Titanium Dioxide Cl 77891), Colorona® Blackstar Blue (3947% by weight muscovite mica (KH₂(AISiO₄)₃), 53-61% by weight Fe₃O₄ (INCl: Iron Oxides Cl 77499)), Colorona® Siena Fine (35-45% by weight muscovite mica (KH₂(AISiO₄)₃), 55-65% by weight Fe₂O₃ (INCl: Iron Oxides Cl 77491)), Colorona® Aborigine Amber (50-62% by weight muscovite mica (KH₂(AISiO₄)₃), 36-44% by weight Fe₂O₃ (INCl: Iron Oxides Cl 77491), 2-6% by weight TiO₂ (INCl: Titanium Dioxide Cl 77891), Colorona® Patagonian Purple (42-54% by weight muscovite mica (KH₂(AlSiO₄)₃), 26-32% by weight Fe₂O₃ (INCl: Iron Oxides Cl 77491), 18-22% by weight TiO₂ (INCl: Titanium Dioxide Cl 77891), 2-4% by weight Prussian Blue (INCl: Ferric Ferrocyanide Cl 77510)), Colorona® Chameleon (40-50% by weight muscovite mica (KH₂(AISiO₄)₃), 50-60% by weight Fe₂O₃ (INCl: Iron Oxides Cl 77491) and Silk® Mica (>98% by weight muscovite mica (KH₂(AISiO₄)₃).

Further particulars of the pearlescent pigments suitable for use in the shaped bodies according to the invention can be found in Inorganic Pigments, Chemical Technology Review No. 166, 1980, pages 161-173 (ISBN 0-8155-0811-5) and Industrial Inorganic Pigments, 2nd Edition, Weinheim, VCH, 1998, pages 211-231, to which reference is expressly made.

The shaped body according to the invention may also contain oxidizing agents. Although, in principle, there are no limits to the choice of the oxidizing agent, it can be of advantage in accordance with the invention to use products of the addition of hydrogen peroxide, more particularly onto urea, melamine or sodium borate, as oxidizing agents. The use of percarbamide is particularly preferred.

Oxidation may also be carried out with enzymes. In this case, the enzymes may be used both to produce oxidizing per compounds and to enhance the effect of an oxidizing agent present in small quantities.

Thus, the enzymes (enzyme class 1: oxidoreductases) are capable of transferring electrons from suitable primary intermediates (reducing agents) to atmospheric oxygen. Preferred enzymes are oxidases, such as tyrosinase and laccase, although glucoseoxidase, uricase or pyruvate oxidase may also be used. Mention is also made of the procedure whereby the effect of small quantities (for example 1% and less, based on the composition as a whole) of hydrogen peroxide is strengthened by peroxidases.

Development of the color may be further supported and enhanced by adding certain metal ions to the shaped body. Examples of such metal ions are Zn²⁺, Cu²⁺, Fe²⁺, Fe³⁺, Mn²⁺, Mn⁴⁺, Li⁺, Mg²⁺, Ca²⁺ and Al³⁺. Zn²⁺, Cu²⁺ and Mn²⁺ are particularly suitable. Basically, the metal ions may be used in the form of a physiologically compatible salt. Preferred salts are the acetates, sulfates, halides, lactates and tartrates. Development of the hair color can be accelerated and the color tone can be influenced as required through the use of these metal salts. However, it has also proved to be practicable to use the metal ions in the form of their complexes or even added onto zeolites to increase coloring power.

In one special embodiment, the tablet according to the invention is free from oxidizing agents.

On noticing the shaped bodies, particularly their spherical shape, optionally in conjunction with aromatic perfume notes, the consumer might associate the colorant according to the invention with a luxury food item, such as confectionery items for example. Through this association, ingestion or rather swallowing of the shaped body, particularly by children, cannot basically be ruled out. In a preferred embodiment, therefore, the shaped bodies according to the invention contain a bitter principle to prevent them from being swallowed or accidentally ingested. According to the invention, preferred bitter principles are those of which at least 5 g/l are soluble in water at 20° C.

So far as unwanted interactions with perfume components optionally present in the shaped body, particularly a change in the perfume note noticed by the consumer, are concerned, ionic bitter principles have proved superior to nonionic types. Accordingly, ionic bitter principles preferably consisting of organic cation(s) and organic anion(s) are preferred for the preparations according to the invention.

According to the invention, quaternary ammonium compounds containing an aromatic group both in the cation and in the anion are eminently suitable as bitter principles. One such compound is benzyl diethyl-((2,6-xylylcarbamoyl)-methyl)-ammonium benzoate which is commercially obtainable, for example, under the names of Bitrex® and Indigestine. This compound is also known by the name of Denatonium Benzoate.

The bitter principle is present in the shaped bodies according to the invention in quantities of 0.0005 to 0.1% by weight, based on the shaped body. Quantities of 0.001 to 0.05% by weight are particularly preferred.

Other components

Besides the ingredients mentioned, the shaped bodies according to the invention may also contain all the known active components, additives and auxiliaries for such preparations. Both solids and liquids may be used as further components. If liquids are selected as further components of the shaped body according to the invention, the quantity used should be selected so that a flowable powder is present before tabletting. The liquid additional components are preferably sprayed onto the powder to be tabletted by suitable means before the tabletting process. Another way of incorporating liquid components in the shaped bodies according to the invention is, for example, to remove solvents beforehand, so that the originally liquid component can be handled as a solid.

In many cases, the shaped bodies contain at least one surfactant. In principle, both anionic and zwitterionic, ampholytic, nonionic and cationic surfactants are suitable. In many cases, however, it has proved to be of advantage to select the surfactants from anionic, zwitterionic or nonionic surfactants.

Suitable anionic surfactants for the preparations according to the invention are any anionic surface-active substances suitable for use on the human body. Such substances are characterized by a water-solubilizing anionic group such as, for example, a carboxylate, sulfate, sulfonate or phosphate group and a lipophilic alkyl group containing around 10 to 22 carbon atoms. In addition, glycol or polyglycol ether groups, ester, ether and amide and hydroxyl groups may also be present in the molecule. The following are examples of suitable anionic surfactants—in the form of the sodium, potassium and ammonium salts and the mono-, di- and trialkanolammonium salts containing 2 or 3 carbon atoms in the alkanol group:
linear fatty acids containing 10 to 22 carbon atoms (soaps),
ether carboxylic acids corresponding to the formula R—O—(CH₂—CH₂O)ₓ—CH₂—COOH, in which R is a linear alkyl group containing 10 to 22 carbon atoms and x=0 or 1 to 16,
acyl sarcosides containing 10 to 18 carbon atoms in the acyl group,
acyl taurides containing 10 to 18 carbon atoms in the acyl group,
acyl isethionates containing 10 to 18 carbon atoms in the acyl group,
sulfosuccinic acid mono- and dialkyl esters containing 8 to 18 carbon atoms in the alkyl group and sulfosuccinic acid monoalkyl polyoxyethyl esters containing 8 to 18 carbon atoms in the alkyl group and 1 to 6 oxyethyl groups,
linear alkane sulfonates containing 12 to 18 carbon atoms,
linear α-olefin sulfonates containing 12 to 18 carbon atoms, α-sulfofatty acid methyl esters of fatty acids containing 12 to 18 carbon atoms,
alkyl sulfates and alkyl polyglycol ether sulfates corresponding to the formula R—O(CH₂—CH₂O)ₓ—SO₃H, in which R is a preferably linear alkyl group containing 10 to 18 carbon atoms and x=0 or 1 to 12, mixtures of surface-active hydroxysulfonates according to DE-A-37 25 030,
sulfated hydroxyalkyl polyethylene and/or hydroxyalkylene propylene glycol ethers according to DE-A-37 23 354,
sulfonates of unsaturated fatty acids containing 12 to 24 carbon atoms and 1 to 6 double bonds according to DE-A-39 26 344,
esters of tartaric acid and citric acid with alcohols in the form of addition products of around 2 to 15 molecules of ethylene oxide and/or propylene oxide with fatty alcohols containing 8 to 22 carbon atoms.

Preferred anionic surfactants are alkyl sulfates, alkyl polyglycol ether sulfates and ether carboxylic acids containing 10 to 18 carbon atoms in the alkyl group and up to 12 glycol ether groups in the molecule and, in particular, salts of saturated and, more particularly, unsaturated C₈₋₂₂ carboxylic acids, such as stearic acid, oleic acid, isostearic acid and palmitic acid.

Nonionic surfactants contain, for example, a polyol group, a poly-alkylene glycol ether group or a combination of polyol and polyglycol ether groups as the hydrophilic group. Examples of such compounds are
products of the addition of 2 to 30 mol ethylene oxide and/or 0 to 5 mol propylene oxide onto linear fatty alcohols containing 8 to 22 carbon atoms, onto fatty acids containing 12 to 22 carbon atoms and onto alkylphenols containing 8 to 15 carbon atoms in the alkyl group,
C₁₂₋₂₂ fatty acid monoesters and diesters of products of the addition of 1 to 30 mol ethylene oxide onto glycerol,
C₈₋₂₂ alkyl mono- and oligoglycosides and ethoxylated analogs thereof,
products of the addition of 5 to 60 mol ethylene oxide onto castor oil and hydrogenated castor oil.

Preferred nonionic surfactants are alkyl polyglycosides corresponding to the general formula R¹O—(Z)ₓ. These compounds are commercially obtainable from Henkel under the name of Plantacare® and are characterized by the following parameters.

The alkyl group R¹ contains 6 to 22 carbon atoms and may be both linear and branched. Primary linear and 2-methyl-branched aliphatic groups are preferred. Such alkyl groups are, for example, 1-octyl, 1-decyl, 1-lauryl, 1-myristyl, 1-cetyl and 1-stearyl. 1-Octyl, 1-decyl, 1-lauryl and 1-myristyl are particularly preferred. Where so-called “oxo alcohols” are used as starting materials, compounds with an odd number of carbon atoms in the alkyl chain predominate.

The alkyl polyglyosides suitable for use in accordance with the invention may, for example, contain only one particular alkyl group R¹. However, such compounds are normally prepared from natural fats and oils or mineral oils. In this case, mixtures corresponding to the starting compounds or corresponding to the particular working up of these compounds are present as the alkyl groups R¹.

Particularly preferred alkyl polyglycosides are those in which R¹ consists
essentially of C₈ and C₁₀ alkyl groups,
essentially of C₁₂ and C₁₄ alkyl groups,
essentially of C₈ to C₁₆ alkyl groups or
essentially of C₁₂ to C₁₆ alkyl groups.

Any mono- or oligosaccharides may be used as the sugar unit Z. Sugars containing 5 or 6 carbon atoms and the corresponding oligosaccharides are normally used. Examples of such sugars are glucose, fructose, galactose, arabinose, ribose, xylose, lyxose, allose, altrose, mannose, gulose, idose, talose and sucrose. Preferred sugar units are glucose, fructose, galactose, arabinose and sucrose; glucose is particularly preferred.

The alkyl polyglycosides suitable for use in accordance with the invention contain on average 1.1 to 5 sugar units. Alkyl polyglycosides with x values of 1.1 to 1.6 are preferred. Alkyl glycosides where x is 1.1 to 1.4 are most particularly preferred.

Besides acting as surfactants, the alkyl glycosides may also be used to improve the fixing of perfume components to the hair. Accordingly, in cases where the effect of the perfume oil on the hair is intended to last longer than the duration of the hair treatment, alkyl glycosides will preferably be used as another ingredient of the preparations according to the invention. An alkyl glucoside particularly preferred for the purposes of the invention is the commercial product Plantacare® 1200 G.

Alkoxylated homologs of the alkyl polyglycosides mentioned may also be used in accordance with the invention. These homologs may contain on average up to 10 ethylene oxide and/or propylene oxide units per alkyl glycoside unit.

Zwitterionic surfactants may also be used, particularly as co-surfactants. In the context of the invention, zwitterionic surfactants are surface-active compounds which contain at least one quaternary ammonium group and at least one —COO⁽⁻⁾ or —SO₃⁽⁻⁾ group in the molecule. Particularly suitable zwitterionic surfactants are the so-called betaines, such as N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. A preferred zwitterionic surfactant is the fatty acid amide derivative known by the INCl name of Cocamidopropyl Betaine.

Also suitable, particularly as co-surfactants, are ampholytic surfactants. Ampholytic surfactants are surface-active compounds which, in addition to a C₈₋₁₈ alkyl or acyl group, contain at least one free amino group and at least one —COOH or —SO₃H group in the molecule and which are capable of forming inner salts. Examples of suitable ampholytic surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids containing around 8 to 18 carbon atoms in the alkyl group. Particularly preferred ampholytic surfactants are N-cocoalkyl aminopropionate, cocoacyl aminoethyl aminopropionate and C₁₂₋₁₈ acyl sarcosine.

According to the invention, the cationic surfactants used are in particular those of the quaternary ammonium compound, esterquat and amidoamine type.

Preferred quaternary ammonium compounds are ammonium halides, more particularly chlorides and bromides, such as alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride and the imidazolium compounds known under the INCl names of Quaternium-27 and Quaternium-83. The long alkyl chains of the above-mentioned surfactants preferably contain 10 to 18 carbon atoms.

Esterquats are known substances which contain both at least one ester function and at least one quaternary ammonium group as structural element. Preferred esterquats are quaternized ester salts of fatty acids with triethanolamine, quaternized ester salts of fatty acids with diethanol alkylamines and quaternized ester salts of fatty acids with 1,2-dihydroxypropyl dialkylamines. Such products are marketed, for example, under the names of Stepantex®, Dehyquart® and Armocare®. The products Armocare® VGH-70, an N,N-bis-(2-palmitoyloxyethyl)-dimethyl ammonium chloride, and Dehyquart® F-75 and Dehyquart® AU-35 are examples of such esterquats.

The alkyl amidoamines are normally prepared by amidation of natural or synthetic fatty acids and fatty acid cuts with dialkyl aminoamines. A compound from this group particularly suitable for the purposes of the invention is the stearamidopropyl dimethylamine obtainable under the name of Tegoamid® S 18.

Other cationic surfactants suitable for use in accordance with the invention are the quaternized protein hydrolyzates.

Also suitable for use in accordance with the invention are cationic silicone oils such as, for example, the commercially available products Q2-7224 (manufacturer: Dow Corning; a stabilized trimethyl silyl amodimethi-cone), Dow Corning® 929 Emulsion (containing a hydroxylamino-modified silicone which is also known as amodimethicone), SM-2059 (manufacturer: General Electric), SLM-55067 (manufacturer: Wacker) and Abil®-Quat 3270 and 3272 (manufacturer: Th. Goldschmidt; diquaternary polydimethyl siloxanes, Quaternium-80).

One example of a quaternary sugar derivative suitable for use as a cationic surfactant is the commercially available product Glucquat®100 (INCl name: Lauryl Methyl Gluceth-10 Hydroxypropyl Dimonium Chloride).

The compounds containing alkyl groups used as surfactants may be single compounds. In general, however, these compounds are produced from native vegetable or animal raw materials so that mixtures with different alkyl chain lengths dependent upon the particular raw material are obtained.

The surfactants representing addition products of ethylene and/or propylene oxide with fatty alcohols or derivatives of these addition products may be both products with a “normal” homolog distribution and products with a narrow homolog distribution. Products with a “normal” homolog distribution are mixtures of homologs which are obtained in the reaction of fatty alcohol and alkylene oxide using alkali metals, alkali metal hydroxides or alkali metal alcoholates as catalysts. By contrast, narrow homolog distributions are obtained when, for example, hydrotalcites, alkaline earth metal salts of ether carboxylic acids, alkaline earth metal oxides, hydroxides or alcoholates are used as catalysts. The use of products with a narrow homolog distribution can be of advantage.

In addition, the shaped bodies according to the invention may preferably contain another conditioning agent selected from the group consisting of cationic surfactants, cationic polymers, alkyl amidoamines, paraffin oils, vegetable oils and synthetic oils. So far as the cationic surfactants are concerned, reference is made to the foregoing observations.

Preferred conditioning agents include cationic polymers. These are generally polymers which contain a quaternary nitrogen atom, for example in the form of an ammonium group.

Preferred cationic polymers are, for example,
The quaternized cellulose derivatives commercially available under the names of Celquat® and Polymer JR®. The compounds Celquat® H 100, Celquat® L 200 and Polymer JR® 400 are preferred quaternized cellulose derivatives.
Polymeric dimethyl diallyl ammonium salts and copolymers thereof with acrylic acid and with esters and amides of acrylic acid and methacrylic acid. The products commercially available under the names of Merquat®100 (poly(dimethyl diallyl ammonium chloride)), Merquat®550 (dimethyl diallyl ammonium chloride/acrylamide copolymer) and Merquat® 280 (dimethyl diallyl ammonium chloride/acrylic acid copolymer) are examples of such cationic polymers.
Copolymers of vinyl pyrrolidone with quaternized derivatives of dialkylaminoacrylate and methacrylate such as, for example, vinyl pyrrolidone/dimethylaminoethyl methacrylate copolymers quaternized with diethyl sulfate. Such compounds are commercially available under the names of Gafquat® 734 and Gafquat® 755,
The vinyl pyrrolidone/methoimidazolinium chloride copolymers commercially available under the name of Luviquat®.
Quaternized polyvinyl alcohol;
and the polymers containing quaternary nitrogen atoms in the main polymer chain known under the names of Polyquaternium 2, Polyquaternium 17, Polyquaternium 18 and Polyquaternium 27.

Cationic polymers belonging to the first four groups mentioned are particularly preferred; Polyquaternium 2, Polyquaternium 10 and Polyquaternium 22 are most particularly preferred.

Other suitable conditioning agents are silicone oils, more particularly dialkyl and alkylaryl siloxanes, such as for example dimethyl polysiloxane and methylphenyl polysiloxane, and alkoxylated and quaternized analogs thereof. Examples of such silicones are the products marketed by Dow Corning under the names of DC 190, DC 200, DC 344, DC 345 and DC 1401 and the products Q2-7224 (manufacturer: Dow Corning; a stabilized trimethyl silyl amodimethicone), Dow Corning® 929 Emulsion (containing a hydroxylamino-modified silicone which is also known as amodimethicone), SM-2059 (manufacturer: General Electric), SLM-55067 (manufacturer: Wacker) and Abil® Quat 3270 and 3272 (manufacturer: Th. Goldschmidt; diquaternary polydimethyl siloxanes, Quaternium-80).

Other suitable conditioning agents are paraffin oils, synthetically produced oligomeric alkenes and vegetable oils, such as jojoba oil, sunflower oil, orange oil, almond oil, wheatgerm oil and peach kernel oil.

Phospholipids, for example soya lecithin, egg lecithin and kephalins, are also suitable hair-conditioning compounds.

In addition, the preparations used in accordance with the invention preferably contain at least one oil component.

Oil components suitable for the purposes of the invention are, in principle, any water-insoluble oils and fatty compounds and mixtures thereof with solid paraffins and waxes. According to the invention, water-insoluble substances are defined as substances of which less than 0.1% by weight dissolves in water at 20° C.

A preferred group of oil components are vegetable oils. Examples of such oils are sunflower oil, olive oil, soya oil, rapeseed oil, almond oil, jojoba oil, orange oil, wheatgerm oil, peach kernel oil and the liquid fractions of coconut oil.

However, other triglyceride oils, such as the liquid fractions of bovine tallow, and synthetic triglyceride oils are also suitable.

Another group of compounds particularly preferred for use as oil components in accordance with the invention are liquid paraffin oils and synthetic hydrocarbons and di-n-alkyl ethers containing a total of 12 to 36 carbon atoms and, more particularly, 12 to 24 carbon atoms, such as for example di-n-octyl ether, di-n-decyl ether, di-n-nonyl ether, di-n-undecyl ether, di-n-doecyl ether, n-hexyl-n-octyl ether, n-octyl-n-decyl ether, n-decyl-n-undecyl ether, n-undecyl-n-dodecyl ether and n-hexyl-n-undecyl ether and ditert.butyl ether, diisopentyl ether, di-3-ethyldecyl ether, tert.butyl-n-octyl ether, isopentyl-n-octyl ether and 2-methylpentyl-n-octyl ether. The compounds 1,3-di-(2-ethylhexyl)-cyclohexane and di-n-octyl ether obtainable as commercial products (Cetiol® S and Cetiol® OE, respectively) can be preferred.

Other oil components suitable for use in accordance with the invention are fatty acid and fatty alcohol esters. The monoesters of fatty acids with alcohols containing 3 to 24 carbon atoms are preferred. This group of substances are products of the esterification of fatty acids containing 6 to 24 carbon atoms such as, for example, caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and the technical mixtures thereof obtained, for example, in the pressure hydrolysis of natural fats and oils, in the reduction of aldehydes from Roelen's oxosynthesis or in the dimerization of unsaturated fatty acids with alcohols such as, for example, isopropyl alcohol, caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof obtained, for example, in the high-pressure hydrogenation of technical methyl esters based on fats and oils or aldehydes from Roelen's oxosynthesis and as monomer fraction in the dimerization of unsaturated fatty alcohols. According to the invention, isopropyl myristate, isononanoic acid C₁₆₋₁₈ alkyl ester (Cetiol® SN), stearic acid-2-ethylhexyl ester (Cetiol®868), cetyl oleate, glycerol tricaprylate, cocofatty alcohol caprate/caprylate and n-butyl stearate are particularly preferred.

Other oil components suitable for use in accordance with the invention are dicarboxylic acid esters, such as di-n-butyl adipate, di-(2-ethylhexyl)-adipate, di-(2-ethylhexyl)-succinate and diisotridecyl acelate, and diol esters, such as ethylene glycol dioleate, ethylene glycol diisotridecanoate, propylene glycol di-(2-ethylhexanoate), propylene glycol diisostearate, propylene glycol dipelargonate, butanediol diisostearate and neopentyl glycol dicaprylate, and also complex esters, for example diacetyl glycerol monostearate.

Finally, fatty alcohols containing 8 to 22 carbon atoms may also be used as oil components in accordance with the invention. The fatty alcohols may be saturated or unsaturated and linear or branched. Examples of fatty alcohols suitable for use in accordance with the invention are decanol, octanol, octenol, dodecenol, decenol, octadienol, dodecadienol, decadienol, oleyl alcohol, erucyl alcohol, ricinolyl alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol and Guerbet alcohols thereof (this list is purely exemplary and is not intended to limit the invention in any way). However, the fatty alcohols emanate from preferably natural fatty acids, normally being obtained from the esters of the fatty acids by reduction. According to the invention, it is also possible to use the fatty alcohol cuts which are produced by reduction of naturally occurring triglycerides, such as bovine tallow, palm oil, peanut oil, rapeseed oil, cottonseed oil, soybean oil, sunflower oil and linseed oil, or fatty acid esters formed from the transesterification products thereof with corresponding alcohols and which therefore represent a mixture of different fatty alcohols.

The oil components are used in the shaped bodies according to the invention in quantities of preferably 0.05 to 10% by weight and more particularly 0.1 to 2% by weight.

In a preferred embodiment of the present invention, a gel is formed as the shaped bodies dissolve in water. To this end, thickeners are added to the shaped body in the form of agar agar, guar gum, alginates, xanthan gum, gum arabic, karaya gum, locust bean gum, linseed gums, dextrans, cellulose derivatives, for example methyl cellulose, hydroxyalkyl cellulose and carboxymethyl cellulose, starch fractions and derivatives, such as amylose, amylopectin and dextrins, clays such as bentonite for example, the silicates marked, for example, under the names of Optigel® (Süd-Chemie) or Laponite® (Solvay) or fully synthetic hydrocolloids, such as polyvinyl alcohol, for example. Particularly preferred thickeners are xanthans, alginates and highly substituted carboxymethyl celluloses.

Other active substances, auxiliaries and additives are, for example,
zwitterionic and amphoteric polymers such as, for example, acrylamido-propyl/trimethyl ammonium chloride/acrylate copolymers and octyl acrylamide/methyl methacrylate/tert.butyl aminoethyl methacrylate/2-hydroxypropyl methacrylate copolymers,
anionic polymers such as, for example, polyacrylic acids, crosslinked polyacrylic acids, vinyl acetate/crotonic acid copolymers, vinyl pyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert.butyl acrylamide terpolymers,
structurants, such as maleic acid and lactic acid,
protein hydrolyzates, more particularly elastin, collagen, keratin, milk protein, soya protein and wheat protein hydrolyzates, condensation products thereof with fatty acids and quaternized protein hydrolyzates,
perfume oils, dimethyl isosorbide and cyclodextrins,
solvents and solubilizers, such as ethylene glycol, propylene glycol, glycerol and diethylene glycol,
fiber-structure-improving agents, more particularly mono-, di- and oligosaccharides such as, for example, glucose, galactose, fructose and lactose,
quaternized amines, such as methyl-1-alkylamidoethyl-2-alkylimidazolinium methosulfate,
defoamers, such as silicones,
dyes for coloring the preparation,
antidandruff agents, such as piroctone olamine, zinc omadine and climbazol,
UV filters, more particularly derivatized benzophenones, cinnamic acid derivatives and triazines,
substances for adjusting the pH value, for example typical acids, more particularly food-grade acids and bases,
active substances, such as allantoin, pyrrolidone carboxylic acids and salts thereof and bisabolol,
vitamins, provitamins and vitamin precursors, more particularly those of groups A, B₃, B₅,B₆, C, E, F and H,
plant extracts, such as the extracts of green tea, oak bark, stinging nettle, hamamelis, hops, camomile, burdock root, horse willow, hawthorn, lime blossom, almond, aloe vera, pine needle, horse chestnut, sandalwood, juniper, coconut, mango, apricot, lemon, wheat, kiwi, melon, orange, grapefruit, sage, rosemary, birch, mallow, lady's smock, creeping thyme, yarrow, thyme, balm, restharrow, coltsfoot, hibiscus, meristem, ginseng and ginger root,
cholesterol,
consistency factors, such as sugar esters, polyol esters or polyol alkyl ethers,
fats and waxes, such as spermaceti, beeswax, montan wax and paraffins,
fatty acid alkanolamides,
complexing agents, such as EDTA, NTA, β-alanine diacetic acid and phosphonic acids,
swelling and penetration agents, such as glycerol, propylene glycol monoethyl ether, carbonates, hydrogen carbonates, guanidines, ureas and primary, secondary and tertiary phosphates,
opacifiers, such as latex, styrene/PVP and styrene/acrylamide copolymers,
pearlizers, such as ethylene glycol mono- and distearate and PEG-3-distearate,
stabilizers for the oxidizing agent,
antioxidants.
   Geometries of the Shaped Body

The shaped bodies according to the invention may assume any geometric form such as, for example, concave, convex, biconcave, biconvex, cubic, tetragonal, orthorhombic, cylindrical, spherical, cylinder-segment-like, disk-shaped, tetrahedral, dodecahedral, octahedral, conical, pyramidal, ellipsoidal, pentagonal-, heptagonal- and hexagonal-prismatic and rhombohedral forms. Completely irregular bases, such as arrow and animal shapes, trees, clouds etc. can also be produced. According to the invention, preferred shapes are slabs, bars, cubes, squares and corresponding shapes with flat sides and, in particular, cylindrical forms of circular or oval cross-section and spherical shaped bodies. Substantially spherical shaped bodies are particularly preferred.

The cylindrical geometry encompasses shapes from tablets to compact cylinders with a height-to-diameter ratio of more than 1. If the basic shaped body has corners and edges, they are preferably rounded off. As an additional optical differentiation, an embodiment with rounded-off corners and bevelled (“chamfered”) edges is preferred.

Besides a spherical shape per se, the spherical geometry also encompasses a sphere/cylinder hybrid where each base of the cylinder is capped by a hemisphere. In this embodiment, the hemispheres preferably have a radius of ca. 4 mm while the shaped body as a whole has a length of 12 to 14 mm.

A spherical shaped body according to the invention may be produced by known processes. The shaped body may be produced by extrusion and subsequent shaping/forming of a premix, as described in detail, for example, in WO-A-91/02047 to which reference is expressly made in the present application.

Accordingly, in another preferred embodiment, substantially spherical shaped bodies are produced in particular by extrusion and subsequent rounding for shaping/forming.

In another embodiment, the portioned pressings may be formed as separate individual elements which correspond to a predetermined dose of the oxidation dye precursor of the secondary intermediate type. However, it is also possible to form pressings which combine several such units in a single pressing, smaller portioned units being easy to break off in particular through the provision of predetermined weak spots. It can be of advantage to produce the portioned pressings as cylindrical or square tablets, preferably with a diameter-to-height ratio of about 0.5:2 to 2:0.5. Commercially available hydraulic presses, eccentric presses and rotary presses are particularly suitable for the production of pressings such as these.

Another possible shape for the shaped body according to the invention has a rectangular base, the height of the shaped body being smaller than the smaller side of the rectangular base. Rounded-off corners are preferred for this supply form.

Another shaped body which can be produced has a plate-like or slab-like structure with alternately thick long segments and thin short segments, so that individual segments can be broken off from this “bar” at the predetermined weak spots, which the short thin segments represent, and introduced into the machine. This “bar” principle can also be embodied in other geometric forms, for example vertical triangles which are only joined to one another at one of their longitudinal sides.

If the shaped bodies according to the invention contain at least one other component besides the secondary intermediate, it can be of advantage in another embodiment not to compress the various components to form a single tablet. In this embodiment, the tabletting process gives shaped bodies comprising several layers, i.e. at least two layers. These various layers may have different dissolving rates. This can provide the shaped bodies with favorable performance properties. If, for example, the shaped bodies contain components which adversely affect one another, one component may be integrated in the more quickly dissolving layer while the other component may be incorporated in a more slowly dissolving layer so that the first component can already have reacted off by the time the second component dissolves.

The various layers of the shaped body can be arranged in the form of a stack, in which case the inner layer(s) dissolve at the edges of the shaped body before the outer layers have completely dissolved. In the stack-like arrangement, the axis of the stack may be arranged as required in relation to the axis of the tablet. Accordingly, in the case of a cylindrical tablet for example, the axis of the stack may run parallel to or perpendicularly of the height of the cylinder.

In another preferred embodiment, however, the inner layer(s) may also be completely surrounded by the layers lying further to the outside which prevents constituents of the inner layer(s) from dissolving prematurely. Shaped bodies where the layers containing the various active components surround one another are preferred. For example, a layer (A) is completely surrounded by layer (B) which is turn is completely surrounded by layer (C). In other preferred shaped bodies, for example, layer (C) is completely surrounded by layer (B) which in turn is completely surrounded by layer (A).

Similar effects can also be obtained by coating of individual constituents of the composition to be tabletted or the shaped body as a whole. To this end, the components to be coated may be sprayed, for example, with aqueous solutions or emulsions or may be coated by the process known as melt coating. For example, the use of a coating of hydroxypropyl methyl cellulose, cellulose, PEG stearates and pigments has been found to be suitable for the purposes of the invention.

As described above, the (recess) tablets produced in accordance with the invention may be completely or partly coated. Processes in which an aftertreatment comprises applying a coating to those surfaces of the shaped body where the filled recess(es) are situated or applying a coating to the shaped body as a whole are preferred for the purposes of the invention.

The shaped body according to the invention has a fracture hardness of preferably 30 to 100 N, more preferably 40 to 80 N and most preferably 50 to 60 N (as measured to the Europäisches Arzneibuch 1997, 3rd Edition, ISBN 3-7692-2186-9, “2.9.8. Bruchfestigkeit von Tabletten (Fracture Resistance of Tablets)”; pages 143-144, using a Schleuniger 6D tablet hardness tester).

In addition, the shaped bodies according to the invention may consist of a shaped body with a recess (known as the “basic tablet”) produced by known tabletting processes. In this embodiment, the basic tablet is produced first and the other compressed part is applied to or introduced into the basic tablet in another step. The resulting product is generally referred to hereinafter as a “recess shaped body” or “recess tablet”.

According to the invention, the basic tablet may in principle assume any practicable shape. The shapes mentioned above are particularly preferred. The shape of the recess may be freely selected, shaped bodies according to the invention in which at least one recess may assume a concave, convex, cubic, tetragonal, orthorhombic, cylindrical, spherical, cylinder-segment-like, disk-shaped, tetrahedral, dodecahedral, octahedral, conical, pyramidal, ellipsoidal, pentagonal-, heptagonal- and hexagonal-prismatic and rhombohedral form being preferred. The recess may also assume a totally irregular shape, such as arrow or animal shapes, trees, clouds etc. As with the basic tablets, recesses with rounded-off corners and edges or with rounded-off corners and chamfered edges are preferred.

The size of the recess by comparison with the shaped body as a whole is governed by the application envisaged for the shaped bodies. The size of the recess can vary according to whether the second compressed part is intended to contain a relatively small or relatively large amount of active component. Irrespective of the intended application, preferred shaped bodies are characterized in that the ratio by weight of the basic tablet to the recess filling is in the range from 1:1 to 100:1, preferably in the range from 2:1 to 80:1, more preferably in the range from 3:1 to 50:1 and most preferably in the range from 4:1 to 30:1.

Similar observations may be made on the contributions made by the basic tablet and the recess filling to the total surface of the shaped body. In preferred shaped bodies, the surface of the pressed-in recess filling makes up 1 to 25%, preferably 2 to 20%, more preferably 3 to 15% and most preferably 4 to 10% of the total surface of the filled basic tablet.

If, for example, the shaped body as a whole has dimensions of 20×20×40 mm and, hence, a total surface area of 40 cm², preferred recess fillings have a surface area of 0.4 to 10 cm², preferably 0.8 to 8 cm², more preferably 1.2 to 6 cm² and most preferably 1.6 to 4 cm².

The recess filling and the basic tablet are preferably colored for optical differentiation. Besides this optical differentiation, recess tablets have performance-related advantages on the one hand through different solubilities of the various regions and, on the other hand, through the separate storage of the active components in the various regions of the shaped body.

According to the invention, shaped bodies where the pressed-in recess filling dissolves more slowly than the basic tablet are preferred. The incorporation of certain components on the one hand enables the solubility of the recess filling to be varied as required; on the other hand, the release of certain ingredients from the recess filling can lead to advantages in the coloring process. Ingredients which, preferably, are at least partly located in the recess filling are, for example, the conditioning components, oil components, vitamins and vegetable active components described under the heading of “other components.”

Tabletting

In a preferred embodiment of the invention, individual active components may be separately encapsulated before incorporation in the shaped tablet. For example, particularly reactive components or even the perfumes may be used in encapsulated form.

The shaped bodies according to the invention are produced by first dry-mixing the ingredients—which may be completely or partly pregranulated—and then shaping/forming, more particularly tabletting, the resulting mixture using conventional processes. To produce the tablets according to the invention, the premix is compacted between two punches in a die to form a solid compactate. This process, which is referred to in short hereinafter as tabletting, comprises four phases, namely metering, compacting (elastic deformation), plastic deformation and ejection.

The premix is first introduced into the die, the filling level and hence the weight and shape of the shaped body formed being determined by the position of the lower punch and the shape of the die. Uniform dosing, even at high tablet throughputs, is preferably achieved by volumetric dosing of the premix. As the tabletting process continues, the top punch comes into contact with the premix and continues descending towards the bottom punch. During this compaction phase, the particles of the premix are pressed closer together, the void volume in the filling between the punches continuously diminishing. The plastic deformation phase in which the particles coalesce and form the shaped body begins from a certain position of the top punch (and hence from a certain pressure on the premix). Depending on the physical properties of the premix, its constituent particles are also partly crushed, the premix sintering at even higher pressures. As the tabletting rate increases, i.e. at high throughputs, the elastic deformation phase becomes increasingly shorter so that the shaped bodies formed can have more or less large voids. In the final step of the tabletting process, the shaped body is forced from the die by the bottom punch and carried away by following conveyors. At this stage, only the weight of the shaped body is definitively established because the tablets can still change shape and size as a result of physical processes (re-elongation, crystallographic effects, cooling, etc.).

The tabletting process is carried out in commercially available tablet presses which, in principle, may be equipped with single or double punches. In the latter case, not only is the top punch used to build up pressure, the bottom punch also moves towards the top punch during the tabletting process while the top punch presses downwards. For small production volumes, it is preferred to use eccentric tablet presses in which the punch(es) is/are fixed to an eccentric disc which, in turn, is mounted on a shaft rotating at a certain speed. The movement of these punches is comparable with the operation of a conventional four-stroke engine. Tabletting can be carried out with a top punch and a bottom punch, although several punches can also be fixed to a single eccentric disc, in which case the number of die bores is correspondingly increased. The throughputs of eccentric presses vary according to type from a few hundred to at most 3,000 tablets per hour.

For larger throughputs, rotary tablet presses are generally used. In rotary tablet presses, a relatively large number of dies is arranged in a circle on a so-called die table. The number of dies varies—according to model—between 6 and 55, although even larger dies are commercially available. Top and bottom punches are associated with each die on the die table, the tabletting pressures again being actively built up not only by the top punch or bottom punch, but also by both punches. The die table and the punches move about a common vertical axis, the punches being brought into the filling, compaction, plastic deformation and ejection positions by means of curved guide rails. At those places where the punches have to be raised or lowered to a particularly significant extent (filling, compaction, ejection), these curved guide rails are supported by additional push-down members, pull-down rails and ejection paths. The die is filled from a rigidly arranged feed unit, the so-called filling shoe, which is connected to a storage container for the premix. The pressure applied to the premix can be individually adjusted through the tools for the top and bottom punches, pressure being built up by the rolling of the punch shank heads past adjustable pressure rollers.

To increase throughput, rotary presses can also be equipped with two filling shoes so that only half a circle has to be negotiated to produce a tablet. To produce two-layer or multiple-layer tablets, several filling shoes are arranged one behind the other without the lightly compacted first layer being ejected before further filling. Given suitable process control, shell and bull's-eye tablets—which have a structure resembling an onion skin—can also be produced in this way. In the case of bull's-eye tablets, the upper surface of the core or rather the core layers is not covered and thus remains visible. Rotary tablet presses can also be equipped with single or multiple punches so that, for example, an outer circle with 50 bores and an inner circle with 35 bores can be simultaneously used for tabletting. Modern rotary tablet presses have throughputs of more than one million tablets per hour.

Where rotary presses are used for tabletting, it has proved to be of advantage to carry out the tabletting process with minimal variations in the weight of the tablets. Variations in tablet hardness can also be reduced in this way. Minimal variations in weight can be achieved as follows:
using plastic inserts with minimal thickness tolerances
low rotor speed
large filling shoe
adapting the rotational speed of the filling shoe blade to the rotor speed
filling shoe with constant powder height
decoupling the filling shoe from the powder supply

Any of the nonstick coatings known in the art may be used to reduce caking on the punch. Plastic coatings, plastic inserts or plastic punches are particularly advantageous. Rotating punches have also proved to be of advantage; if possible, the upper and lower punches should be designed for rotation. If rotating punches are used, there will generally be no need for a plastic insert. In that case, the surfaces of the punch should be electropolished.

It has also been found that long tabletting times are advantageous. These can be achieved by using pressure rails, several pressure rollers or low rotor speeds. Since variations in tablet hardness are caused by variations in the pressures applied, systems which limit the tabletting pressure should be used. Elastic punches, pneumatic compensators or spring elements in the force path may be used. The pressure roller can also be spring-mounted.

Tabletting machines suitable for the purposes of the invention can be obtained, for example, from the following companies: Apparatebau Holzwarth GbR, Asperg; Wilhelm Fette GmbH, Schwarzenbek; Fann Instruments Company, Houston, Texas (USA); Hofer GmbH, Weil; Horn & Noack Pharmatechnik GmbH, Worms; IMA Verpackungssysteme GmbH Viersen; KILIAN, Cologne; KOMAGE, Kell am See, KORSCH Pressen GmbH, Berlin; and Romaco GmbH, Worms. Other suppliers are, for example Dr. Herbert Pete, Vienna (AU); Mapag Maschinenbau AG, Bern (Switzerland); BWI Manesty, Liverpool (GB); I. Holand Ltd., Nottingham (GB); and Courtoy N. V., Halle (BE/LU) and Medicopharm, Kamnik (SI). One example of a particularly suitable tabletting machine is the model HPF 630 hydraulic double-pressure press manufactured by LAEIS, D. Tabletting tools are obtainable, for example, from Adams Tablettierwerkzeuge Dresden; Wilhelm Feft GmbH, Schwarzenbek; Klaus Hammer, Solingen; Herber & Söhne GmbH, Hamburg; Hofer GmbH, Weil; Horn & Noack, Pharmatechnik GmbH, Worms; Ritter Pharmatechnik GmbH, Hamburg; Romaco GmbH, Worms and Notter Werkzeugbau, Tamm. Other suppliers are, for example, Senss AG, Reinach (CH) and Medicopharm, Kamnik (SI).

However, the process for producing the shaped bodies is not confined to compressing just one particulate premix to form a shaped body. Instead, the process may also be augmented to the extent that multilayer shaped bodies are produced in known manner by preparing two or more premixes which are pressed onto one another. In this case, the first premix introduced is lightly precompressed in order to obtain a smooth upper surface running parallel to the base of the shaped body and, after the second premix has been introduced, the whole is compressed to form the final shaped body. In the case of shaped bodies with three or more layers, each addition of premix is followed by further precompression before the shaped body is compressed for the last time after addition of the last premix.

The pressing of the particulate composition into the recess may be carried out similarly to the production of the basic tablet in tablet presses. In a preferred procedure, the basic tablet with recess is first produced, then filled and subsequently re-compressed. This can be done by ejecting the basic tablet from the first tablet press, filling and transporting into a second tablet press in which final compression takes place. Alternatively, final compression may also be carried out by pressure rollers which roll over the shaped bodies on a conveyor belt. However, a rotary tablet press could also be provided with different punch sets, so that a first punch set presses recesses into the shaped bodies while the second punch set, after filling, provides the shaped bodies with a flat surface by re-compression.

In a second embodiment, the present invention relates to a process for coloring keratin-containing fibers which is characterized in that
(I) one or more shaped bodies is/are dissolved in a medium M to form the preparation A,
(II) the resulting preparation A is mixed with an oxidizing agent preparation B to form a ready-to-use colorant,
(III) the colorant F is applied to the fibers and
(IV) is rinsed off again after a contact time.

The preparation A and the oxidizing agent preparation B are mixed in a ratio by weight of preferably about 2:1 to 1:2 and more preferably about 1:1.

It is of course also possible in the process according to the invention to replace the shaped body according to the invention with a powder or granules which, besides a cosmetically acceptable carrier, contain(s) at least one dissolution accelerator and at least one oxidation dye precursor of the secondary intermediate type and is/are free from oxidation dye precursors of the primary intermediate type.

The ready-to-use colorant F should preferably have a pH of 6 to 12. In a particularly preferred embodiment, the hair colorant is application in a weakly alkaline medium. The application temperatures may be in the range from 15 to 40° C. and are preferably the temperatire of the scalp. The contact time is normally ca. 5 to 45 and more particularly 15 to 30 minutes. If the carrier used does not have a high surfactant content, the treated hair may advantageously be cleaned with a shampoo.

In another embodiment, the medium M is preferably a gel or an o/w or w/o emulsion.

The medium M has a viscosity of 500 to 100,000 mPa.s, preferably 3,000 to 70,000 mPa.s, more preferably 6,000 to 50,000 mPa.s and most preferably 10,000 to 30,000 mPa.s. The viscosities are measured with a Brookfield RVT viscosimeter (4 r.p.m., spindle No. 4) at a temperature of 20° C. However, the spindle for measuring the viscosities mentioned is preferably selected according to the viscosity range (as measured under the test conditions mentioned above), as shown in Table 1.

TABLE 1

| Spindle No. | Viscosity range [mPa · s] |
|---|---|
| 1 | −2,500 |
| 2 | >2,500-10,000 |
| 3 | >10,000 to 25,000 |
| 4 | >25,000-50,000 |
| 5 | >50,000 to 100,000 |

In a special embodiment, the medium M has a viscosity of 500 to 50,000 mPa.s, preferably 500 to 25,000 mPa.s and more particularly 500 to 15,000 mPa.s. The viscosities of this special embodiment are measured with a Brookfield RVT viscosimeter (spindle No. 4, 20 r.p.m.) at 20° C.

In a preferred embodiment of the process according to the invention, the medium M contains at least one oxidation dye precursor of the primary intermediate type. According to the invention, the primary intermediate component is preferably a p-phenylenediamine derivatives or one of its physiologically compatible salts. Particularly preferred p-phenylenediamine derivatives correspond to formula (E1): 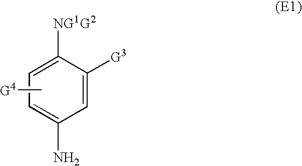
in which
G¹ stands for a hydrogen atom, a C₁₋₄ alkyl radical, a C₁₋₄ monohydroxyalkyl radical, a C₂₋₄ polyhydroxyalkyl radical, a (C₁₋₄)-alkoxy-(C₁₋₄)-alkyl radical, a 4′-aminophenyl radical or a C₁₋₄ alkyl radical substituted by a nitrogen-containing group, a phenyl group or a 4′-aminophenyl group;
G² stands for a hydrogen atom, a C₁₋₄ alkyl radical, a C₁₋₄ monohydroxyalkyl radical, a C₂₋₄ polyhydroxyalkyl radical, a (C₁₋₄)-alkoxy-(C₁₋₄)-alkyl radical or a C₁₋₄ alkyl radical substituted by a nitrogen-containing group;
G³ stands for a hydrogen atom, a halogen atom, such as a chlorine, bromine, iodine or fluorine atom, a C₁₋₄ alkyl radical, a C₁₋₄ monohydroxyalkyl radical, a C₂₋₄ polyhydroxyalkyl radical, a C₁₋₄ hydroxyalkoxy radical, a C₁₋₄ acetylaminoalkoxy radical, a C₁₋₄ mesylaminoalkoxy radical or a C₁₋₄ carbamoylaminoalkoxy radical;
G⁴ is a hydrogen atom, a halogen atom or a C₁₋₄ alkyl radical or
if G³ and G⁴ are in the ortho position to one another, they may together form a bridging α,ω-alkylenedioxo group such as, for example, an ethylenedioxy group.

Examples of the C₁₋₄ alkyl radicals mentioned as substituents in the compounds according to the invention are the methyl, ethyl, propyl, isopropyl and butyl groups. Ethyl and methyl radicals are preferred alkyl radicals. According to the invention, preferred C₁₋₄ alkoxy radicals are, for example, methoxy or ethoxy radicals. Other preferred examples of a C₁₋₄ hydroxyalkyl group are the hydroxymethyl, 2-hydroxyethyl, 3-hydroxypropyl or 4-hydroxybutyl group. A 2-hydroxyethyl group is particularly preferred. A particularly preferred C₂₋₄ polyhydroxyalkyl group is the 1,2-dihydroxyethyl group. According to the invention, examples of halogen atoms are F, Cl or Br atoms. Cl atoms are most particularly preferred. According to the invention, the other terms used are derived from the definitions given here. Examples of nitrogen-containing groups corresponding to formula (E1) are, in particular, the amino groups, C₁₋₄ monoalkylamino groups, C₁₋₄ dialkylamino groups, C₁₋₄ trialkylammonium groups, C₁₋₄ monohydroxyalkylamino groups, imidazolinium and ammonium.

Particularly preferred p-phenylenediamines corresponding to formula (E1) are selected from p-phenylenediamine, p-toluylenediamine, 2-chloro-p-phenylenediamine, 2,3-dimethyl-p-phenylenediamine, 2,6-dimethyl-p-phenylenediamine, 2,6-diethyl-p-phenylenediamine, 2,5-dimethyl-p-phenylenediamine, N,N-dimethyl-p-phenylenediamine, N,N-diethyl-p-phenylenediamine, N,N-dipropyl-p-phenylenediamine, 4-amino-3-methyl-(N,N-diethyl)-aniline, N,N-bis-(β-hydroxyethyl)-p-phenylenediamine, 4-N,N-bis-(β-hydroxyethyl)-amino-2-methylaniline, 4-N,N-bis-(β-hydroxyethyl)-amino-2-chloroaniline, 2-(β-hydroxyethyl)-p-phenylenediamine, 2-(α,β-dihydroxyethyl)-p-phenylenediamine, 2-fluoro-p-phenylenediamine, 2-isopropyl-p-phenylenediamine, N-(β-hydroxypropyl)-p-phenylenediamine, 2-hydroxymethyl-p-phenylenediamine, N,N-dimethyl-3-methyl-p-phenylenediamine, N,N-(ethyl-β-hydroxyethyl)-p-phenylenediamine, N-(β,γ-dihydroxypropyl)-p-phenylenediamine, N-(4′-aminophenyl)-p-phenylenediamine, N-phenyl-p-phenylenediamine, 2-(β-hydroxyethyloxy)-p-phenylenediamine, 2-(β-acetylaminoethyloxy)-p-phenylenediamine, N-(β-methoxyethyl)-p-phenylene-diamine and 5,8-diaminobenzo-1,4-dioxane and physiologically compatible salts thereof.

According to the invention, most particularly preferred p-phenylenediamine derivatives corresponding to formula (E1) are p-phenylenediamine, p-toluylenediamine, 2-(β-hydroxyethyl)-p-phenylene-diamine, 2-(α,β-dihydroxyethyl)-p-phenylenediamine and N,N-bis-(2-hydroxyethyl)-p-phenylenediamine.

In another preferred embodiment of the invention, compounds containing at least two aromatic nuclei substituted by amino and/or hydroxyl groups may be used as the primary intermediate.

The binuclear primary intermediate components which may be used in the coloring compositions according to the invention include in particular compounds corresponding to formula (E2) and physiologically compatible salts thereof: 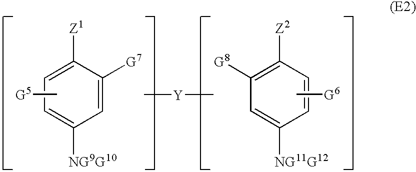
in which
Z¹ and Z² independently of one another stand for a hydroxyl or NH₂ radical which is optionally substituted by a C₁₋₄ alkyl radical, by a C₁₋₄ hydroxyalkyl radical and/or by a bridging group Y or which is optionally part of a bridging ring system,
the bridging group Y is a C₁₋₁₄ alkylene group such as, for example, a linear or branched alkylene chain or an alkylene ring which may be interrupted or terminated by one or more nitrogen-containing groups and/or one or more hetero atoms, such as oxygen, sulfur or nitrogen atoms, and may optionally be substituted by one or more hydroxyl or C₁₋₈ alkoxy radicals, or a direct bond
G⁵ and G⁶ independently of one another stand for a hydrogen or halogen atom, a C₁₋₄ alkyl radical, a C₁₋₄ monohydroxyalkyl radical, a C₂₋₄ polyhydroxyalkyl radical, a C₁₋₄ aminoalkyl radical or a direct bond to the bridging group Y,
G⁷, G⁸, G⁹, G¹⁰,G¹¹ and G¹² independently of one another stand for a hydrogen atom, a direct bond to the bridging group Y or a C₁₋₄ alkyl radical,
   with the provisos that
the compounds of formula (E2) contain only one bridging group Y per molecule and
the compounds of formula (E2) contain at least one amino group bearing at least one hydrogen atom.

According to the invention, the substituents used in formula (E2) are as defined in the foregoing.

Preferred binuclear primary intermediates corresponding to formula (E2) are, in particular, N,N′-bis-(β-hydroxyethyl)-N,N′-bis-(4′-aminophenyl)-1,3-diaminopropanol, N,N′-bis-(β-hydroxyethyl)-N,N′-bis-(4′-aminophenyl)ethylenediamine, N,N′-bis-(4-aminophenyl)-tetramethylene diamine, N,N′-bis-(β-hydroxyethyl)-N,N′-bis-(4′-aminophenyl)-tetramethylene diamine, N,N′-bis-(4-methylaminophenyl)-tetramethylene diamine, N,N′-bis-(ethyl)-N,N′-bis-(4′-amino-3′-methylphenyl)-ethylenediamine, bis-(2-hydroxy-5-aminophenyl)-methane, N,N′-bis-(4′-aminophenyl)-1,4-diazacycloheptane, N,N′-bis-(2-hydroxy-5-aminobenzyl)-piperazine, N-(4′-aminophenyl)-p-phenylenediamine and 1,10-bis-(2′,5′-diaminophenyl)-1,4,7,10-tetraoxadecane and physiologically compatible salts thereof.

Most particularly preferred binuclear primary intermediates corresponding to formula (E2) are N,N′-bis-(β-hydroxyethyl)-N,N′-bis-(4′-aminophenyl)-1,3-diaminopropan-2-ol, bis-(2-hydroxy-5-aminophenyl)-methane, N,N′-bis-(4′-aminophenyl)-1,4-diazacycloheptane and 1,10-bis-(2′,5′-diaminophenyl)-1,4,7,10-tetraoxadecane or a physiologically compatible salt thereof.

In another preferred embodiment of the invention, a p-aminophenol derivative or a physiologically compatible salt thereof is used as the primary intermediate. Particularly preferred p-aminophenol derivatives correspond to formula (E3): 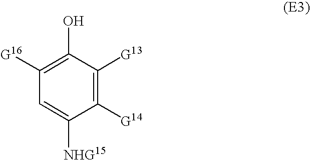
in which
G¹³ stands for a hydrogen atom, a halogen atom, a C₁₋₄ alkyl radical, a C₁₋₄ monohydroxyalkyl radical, a C₂₋₄ polyhydroxyalkyl radical, a (C₁₄)-alkoxy-(C₁₋₄)-alkyl radical, a C₁₋₄ aminoalkyl radical, a hydroxy-(C₁₋₄)-alkylamino radical, a C₁₋₄ hydroxyalkoxy radical, a C₁₋₄ hydroxyalkyl-(C₁₋₄)-aminoalkyl radical or a (di-C₁₋₄-alkylamino)-(C₁₋₄)-alkyl radical,
G¹⁻⁴ stands for a hydrogen atom or a halogen atom, a C₁₋₄ alkyl radical, a C₁₋₄ monohydroxyalkyl radical, a C₂₋₄ polyhydroxyalkyl radical, a (C₁₋₄)-alkoxy-(C₁₋₄)-alkyl radical, a C₁₋₄ aminoalkyl radical or a C₁₋₄ cyanoalkyl radical,
G¹⁵ stands for hydrogen, a C₁₋₄ alkyl radical, a C₁₋₄ monohydroxyalkyl radical, a C₂₋₄ polyhydroxyalkyl radical, a phenyl radical or a benzyl radical and
G¹⁶ stands for hydrogen or a halogen atom.

According to the invention, the substituents used in formula (E3) are defined as in the foregoing.

Preferred p-aminophenols corresponding to formula (E3) are, in particular, p-aminophenol, N-methyl-p-aminophenol, 4-amino-3-methylphenol, 4-amino-3-fluorophenol, 2-hydroxymethylamino-4-aminophenol, 4-amino-3-hydroxymethylphenol, 4-amino-2-(β-hydroxyethoxy)-phenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)-phenol, 4-amino-2-(α,β-dihydroxyethyl)-phenol, 4-amino-2-fluorophenol, 4-amino-2-chlorophenol, 4-amino-2,6-dichlorophenol, 4-amino-2-(diethylaminomethyl)-phenol and physiologically compatible salts thereof.

Most particularly preferred compounds corresponding to formula (E3) are p-aminophenol, 4-amino-3-methylphenol, 4-amino-2-aminomethylphenol, 4-amino-2-(α,β-dihydroxyethyl)-phenol and 4-amino-2-(diethylaminomethyl)-phenol.

In addition, the primary intermediate may be selected from o-aminophenol and its derivatives such as, for example, 2-amino-4-methylphenol, 2-amino-5-methylphenol or 2-amino-4-chlorophenol.

The primary intermediate may also be selected from heterocyclic primary intermediates such as, for example, pyridine, pyrimidine, pyrazole, pyrazole-pyrimidine derivatives and physiologically compatible salts thereof.

Preferred pyridine derivatives are, in particular, the compounds described in GB 1,026,978 and GB 1,153,196, such as 2,5-diaminopridine, 2-(4′-methoxyphenyl)-amino-3-aminopyridine, 2,3-diamino-6-methoxypyridine, 2-(β-methoxyethyl)-amino-3-amino-6-methoxypyridine and 3,4-diaminopyridine.

Preferred pyrimidine derivatives are, in particular, the compounds described in DE 2359399, JP 02019576 A2 and WO 96/15765, such as 2,4,5,6-tetraaminopyrimidine, 4-hydroxy-2,5,6-triaminopyrimidine, 2-hydroxy-4,5,6-triaminopyrimidine, 2-dimethylamino-4,5,6-triaminopyrimidine, 2,4-dihydroxy-5,6-diaminopyrimidine and 2,5,6-triaminopyridine.

Preferred pyrazole derivatives are, in particular, the compounds described in patents DE 3843892 and DE 4133957 and in patent applications WO 94/08969, WO 94/08970, EP 740931 and DE 19543988, such as 4,5-diamino-1-methylpyrazole, 4,5-diamino-1-(β-hydroxyethyl)-pyrazole, 3,4-diaminopyrazole, 4,5-diamino-1-(4′-chlorobenzyl)-pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methyl pyrazole, 4,5-diamino-3-tert.butyl-1-methylpyrazole, 4,5-diamino-1-tert.butyl-3-methylpyrazole, 4,5-diamino-1-(β-hydroxyethyl)-3-methyl-pyrazole, 4,5-diamino-1-ethyl-3-methylpyrazole, 4,5-diamino-1-ethyl-3-(4′-methoxyphenyl)-pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(β-aminoethyl)-amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)-amino-1-methylpyrazole.

Preferred pyrazole-pyrimidine derivatives are, in particular, the derivatives of pyrazole-[1,5-a]-pyrimidine corresponding to formula (E4) below and tautomeric forms thereof where a tautomeric equilibrium exists: 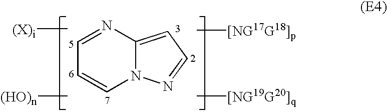
in which
G¹⁷, G¹⁸, G¹⁹ and G²⁰ independently of one another stand for a hydrogen atom, a C₁₋₄ alkyl radical, an aryl radical, a C₁₋₄ hydroxyalkyl radical, a C₂₋₄ polyhydroxyalkyl radical, a (C₁₋₄)-alkoxy-(C₁₋₄)-alkyl radical, a C₁₋₄ aminoalkyl radical which may optionally be protected by an acetylureide or sulfonyl radical, a (C₁₋₄)-alkylamino-(C₁₋₄)-alkyl radical, a di[(C₁₋₄)-alkyl]-(C₁₋₄)-aminoalkyl radical, the dialkyl radicals optionally forming a carbon cycle or a heterocycle with 5 or 6 links, a C₁₋₄ hydroxyalkyl or a di-(C₁₋₄)-[hydroxyalkyl]-(C₁₋₄)-aminoalkyl radical;
the X radicals independently of one another stand for a hydrogen atom, a C₁₋₄ alkyl radical, an aryl radical, a C₁₋₄ hydroxyalkyl radical, a C₂₋₄ polyhydroxyalkyl radical, a C₁₋₄ aminoalkyl radical, a (C₁₋₄)-alkylamino-(C₁₋₄)-alkyl radical, a di[(C₁₋₄)-alkyl]-(C₁₋₄)-aminoalkyl radical, the dialkyl radicals optionally forming a carbon cycle or a heterocycle with 5 or 6 links, a C1-4 hydroxyalkyl or a di-(C₁₋₄)-[hydroxyalkyl]-(C₁₋₄)-aminoalkyl radical, an amino radical, a C₁₋₄ alkyl or a di-(C₁₋₄ hydroxyalkyl)-amino radical, a halogen atom, a carboxylic acid group or a sulfonic acid group,
i has the value 0, 1, 2 or 3,
p has the value 0 or 1,
q has the value 0 or 1 and
n has the value 0 or 1,
   with the proviso that
the sum of p+q is not 0,
where p+q=2, n has the value 0 and the groups NG¹⁷G¹⁸ and NG¹⁹G²⁰ occupy the (2,3); (5,6); (6,7); (3,5) or (3,7) positions;
where p+q=1, n has the value 1 and the groups NG¹⁷G¹⁸ (or NG¹⁹G²⁰) and the group OH occupy the (2,3); (5,6); (6,7); (3,5) or (3,7) positions.

The substituents used in formula (E4) are as defined in the foregoing.

If the pyrazole-[1,5-a]-pyrimidine corresponding to formula (E4) above contains a hydroxy group in one of the positions 2, 5 or 7 of the ring system, a tautomeric equilibrium exists as illustrated, for example, in the following scheme:

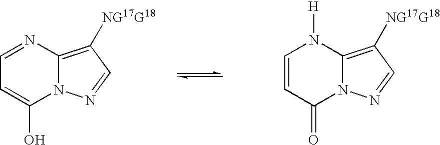

Among the pyrazole-[1,5-a]-pyrimidines corresponding to formula (E4) above, the following may be particularly mentioned:
apyrazole-[1,5-a]-pyrimidine-3,7-diamine;
2,5-dimethylpyrazole-[1,5-a]-pyrimidine-3,7-diamine;
pyrazole-[1,5-a]-pyrimidine-3,5-diamine;
2,7-dimethylpyrazole-[1,5-a]-pyrimidine-3,5-diamine;
3-aminopyrazole-[1,5-a]-pyrimid in-7-ol;
3-aminopyrazole-[1,5-a]-pyrimid in-5-ol;
2-(3-aminopyrazole-[1,5-a]-pyrimidin-7-ylamino)-ethanol;
2-(7-aminopyrazole-[1,5-a]-pyrimidin-3-ylamino)-ethanol;
2-[(3-aminopyrazole-[1,5-a]-pyrimidin-7-yl)-(2-hydroxyethyl)-amino]-ethanol;
2-[(7-aminopyrazole-[1,5-a]-pyrimidin-3-yl)-(2-hydroxyethyl)-amino]-ethanol;
5,6-dimethylpyrazole-[1,5-a]-pyrimidine-3,7-diamine;
2,6-dimethylpyrazole-[1,5-a]-pyrimidine-3,7-diamine;
3-amino-7-dimethylamino-2,5-dimethylpyrazole-[1,5-a]-pyrimidine;
   and physiologically compatible salts thereof and tautomeric forms thereof where a tautomeric equilibrium exists.

The pyrazole-[1,5-a]-pyrimidines corresponding to formula (E4) above may be prepared by cyclization from an aminopyrazole or from hydrazine, as described in the literature.

Besides the primary intermediate components, the medium M may contain at least one secondary intermediate component and/or at least one substantive dye. The secondary intermediate components or substantive dyes preferably used in this embodiment correspond to those mentioned in the foregoing. The observations in the corresponding paragraphs apply.

The oxidation dye precursors or the substantive dyes used in the medium M do not have to be single compounds. On the contrary, other components may be present in small quantities in the shaped bodies according to the invention due to the processes used to produce the individual dyes providing these other components do not adversely affect the coloring result or have to be ruled out for other reasons, for example toxicological reasons.

The oxidation dye precursors are present in the medium M in quantities of preferably 0.01 to 20% by weight and more preferably 0.5 to 5% by weight, based on the medium M as a whole.

Preferred precursors of “nature-analogous” dyes are indoles and indolines which contain at least one hydroxy or amino group, preferably as a substituent on the six ring. These groups may carry further substituents, for example in the form of an etherification or esterification of the hydroxy group or an alkylation of the amino group.

Particularly suitable precursors of “nature-analogous” hair dyes are derivatives of 5,6-dihydroxyindoline corresponding to formula (Ia): 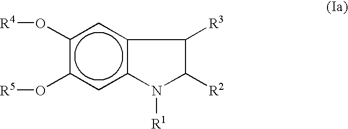
in which—independently of one another—
R¹ is hydrogen, a C₁₋₄ alkyl group or a C₁₋₄ hydroxyalkyl group,
R² is hydrogen or a —COOH group, the —COOH group optionally being present as a salt with a physiologically compatible cation,
R³ is hydrogen or a C₁₋₄ alkyl group,
R⁴ is hydrogen, a C₁₄ alkyl group or a group —CO—R⁶, where R⁶ is a C₁₋₄ alkyl group, and
R⁵ is one of the groups mentioned for R⁴, and physiologically compatible salts of these compounds with an organic or inorganic acid.

Particularly preferred derivatives of indoline are 5,6-dihydroxy-indoline, N-methyl-5,6-dihydroxyindoline, N-ethyl-5,6-dihydroxyindoline, N-propyl-5,6-dihydroxyindoline, N-butyl-5,6-dihydroxyindoline, 5,6-dihydroxy-indoline-2-carboxylic acid and 6-hydroxyindoline, 6-aminoindoline and 4-aminoindoline.

Within this group, particular emphasis is placed on N-methyl-5,6-dihydroxyindoline, N-ethyl-5,6-dihydroxyindoline, N-propyl-5,6-dihydroxy-indoline, N-butyl-5,6-dihydroxyindoline and, in particular, 5,6-dihydroxyindoline.

Other particularly suitable precursors of “nature-analogous” hair dyes are derivatives of 5,6-dihydroxyindole corresponding to formula (Ib): 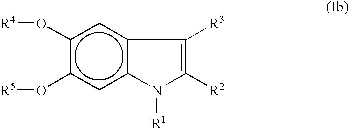
in which—independently of one another—
R¹ is hydrogen, a C₁₋₄ alkyl group or a C₁₋₄ hydroxyalkyl group,
R² is hydrogen or a —COOH group, the —COOH group optionally being present as a salt with a physiologically compatible cation,
R³ is hydrogen or a C₁₋₄ alkyl group,
R⁴ is hydrogen, a C₁₋₄ alkyl group or a group —CO—R⁶, where R⁶ is a C₁₋₄ alkyl group, and
R⁵ is one of the groups mentioned for R⁴,
   and physiologically compatible salts of these compounds with an organic or inorganic acid.

Particularly preferred derivatives of indole are 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, 6-hydroxyindole, 6-aminoindole and 4-aminoindole.

Within this group, particular emphasis is placed on N-methyl-5,6-dihydroxyindole, N-ethyl-5,6-dihydroxyindole, N-propyl-5,6-dihydroxyindole, N-butyl-5,6-dihydroxyindole and, in particular, 5,6-dihydroxyindole.

The indoline and indole derivatives may be used both as free bases and in the form of their physiologically compatible salts with inorganic or organic acids, for example hydrochlorides, sulfates and hydrobromides, in the colorants used in the process according to the invention. The indole or indoline derivatives are present in these colorants in quantities of normally 0.05 to 10% by weight and preferably 0.2 to 5% by weight.

In the particular case where dye precursors of the indoline or indole type are used, it has proved to be of advantage to use as amino acid and/or an oligopeptide as alkalizing agent.

The oxidizing agent preparation B contains at least one oxidizing agent. On the one hand, the oxidizing agent may be used to lighten the fibers to be treated. On the other hand, however, the oxidizing agent may also be used to deveop the actual dye from the dye percursors.

In principle, the color can be oxidatively developed with atmospheric oxygen. However, a chemical oxidizing agent is preferably used, particularly when human hair is to be not only colored, but also lightened. Particularly suitable oxidizing agents are persulfates, chlorites and, in particular, hydrogen peroxide or addition products thereof with urea, melamine or sodium borate. According to the invention, however, the oxidation colorant may also be applied to the hair together with a catalyst which activates the oxidation of the dye precursors, for example by atmospheric oxygen. Such catalysts are, for example, metal ions, iodides, quinones or certain enzymes.

Development of the color may be further supported and enhanced by adding certain metal ions to the shaped body. Examples of such metal ions are Zn²⁺, Cu²⁺, Fe²⁺, Fe³⁺, Mn²⁺, Mn⁴⁺, Li⁺, Mg²⁺, Ca² and Al^{3+. Zn}²⁺, Cu²⁺ and Mn²⁺ are particularly suitable. Basically, the metal ions may be used in the form of a physiologically compatible salt. Preferred salts are the acetates, sulfates, halides, lactates and tartrates. Development of the hair color can be accelerated and the color tone can be influenced as required through the use of these metal salts. However, it has also proved to be practicable to use the metal ions in the form of their complexes or even added onto zeolites to increase coloring power.

Suitable enzymes are, for example, peroxidases which are capable of significantly enhancing the effect of small quantities of hydrogen peroxide. According to the invention, other suitable enzymes are those which directly oxidize the oxidation dye precursors with the aid of atmospheric oxygen, such as the laccases for example, or which produce small quantities of hydrogen peroxide in situ and thus biocatalytically activate the oxidation of the dye precursors. Particularly suitable catalysts for the oxidation of the dye precursors are the so-called 2-electron oxidoreductases in combination with the substrates specific to them, for example
pyranose oxidase and, for example, D-glucose or galactose,
glucose oxidase and D-glucose,
glycerol oxidase and glycerol,
pyruvate oxidase and pyruvic acid or salts thereof,
alcohol oxidase and alcohol (MeOH, EtOH),
lactate oxidase and lactic acid and salts thereof,
tyrosinase oxidase and tyrosine,
uricase and uric acid or salts thereof,
choline oxidase and choline,
amino acid oxidase and amino acids.

Information on other optional components and the quantities in which they are used can be found in the reference books known to the expert, for example Kh. Schrader, Grundlagen und Rezepturen der Kosmetika, 2nd Edition, Hüthig Buch Verlag, Heidelberg, 1989.

In a third embodiment, the present invention relates to the use of the shaped bodies described above for the production of a preparation for coloring keratin fibers.

In a fourth embodiment, the present invention relates to a kit for use in the process according to the invention, characterized in that it contains three separately prepared components in containers K1, K2 and K3, container K1 containing the medium M, container K2 containing one or more shaped bodies according to the invention and container K3 containing the oxidizing agent preparation B.

Packaging of the Shaped Bodies

The shaped bodies according to the invention may be packaged after their production, the use of certain packaging systems having proved to be particularly effective, on the one hand because they increase the storage stability of the ingredients and, on the other hand, because they may also improve the long-term adhesion of a recess filling. In addition, packaging systems increase the protection of the shaped body against destruction by mechanical influences. The term “packaging system” in the context of the present invention always characterizes the primary packaging of the shaped bodies in the container K2, i.e. the pack which is in direct contact on its inside with the surface of the shaped body, Any optional secondary packaging has to meet the usual requirements so that all known materials and systems may be used for this purpose. In a preferred embodiment of the invention, the shaped body is accommodated in a transparent packaging system or this packaging system is optionally packed in transparent secondary packaging.

According to the invention, packaging systems with a low permeability to water vapor are preferred. In this way, the coloring powder of the shaped bodies according to the invention can be maintained over a prolonged period, even if, for example, hygroscopic components are used in the shaped bodies. Particularly preferred packaging systems have a water vapor transmission rate of 0.1 g/m²/day to less than 20 g/m²/day when the packaging system is stored at 23° C./85% relative equilibrium humidity. The temperature and humidity conditions mentioned are the test conditions specified in DIN 53122, according to which minimal deviations are acceptable (23±1° C., 85±2% relative humidity). The water vapor transmission rate of a given packaging system or material can be determined by other standard methods and is also described, for example, in ASTM Standard E-96-53T (“Test for Measuring Water Vapor Transmission of Materials in Sheet Form”) and in TAPPI Standard T464 m-45 (“Water Vapor Permeability of Sheet Materials at High Temperatures and Humidity”). The measurement principle of standard methods is based on the water absorption of anhydrous calcium chloride which is stored in a container in the corresponding atmosphere, the container being closed on top by the material to be tested. The water vapor transmission rate can be calculated from the surface of the container closed by the material to be tested (permeation surface), the increase in weight of the calcium chloride and the exposure time in accordance with the following equation:

$$WVTR = \frac{24 \cdot 10000}{A} \cdot \frac{x}{y} [ g \text{/} {m}^{2} \text{/} 24 h ]$$

where A is the surface area of the material to be tested in cm², x is the increase in weight of the calcium chloride in g and y is the exposure time in h.

The relative equilibrium humidity, often referred to as “relative air humidity”, in the measurement of the water vapor transmission rate for the purposes of the present invention is 85% at 23° C. The absorption capacity of air for water vapor increases with temperature to a particular maximum content, the so-called saturation content, and is expressed in g/m³. For example, 1 m³ of air at 17° is saturated with 14.4 g of water vapor, the saturation content at 11° being as much as 10 g of water vapor. The relative air humidity is the ratio expressed in percent between the water vapor content actually present and the saturation content corresponding to the prevailing temperature. If, for example, air at 17° contains 12 g/m³ water vapor, the relative air humidity is (12/14.4)·100=83%. If this air is cooled, saturation (100% relative humidity) is reached at the so-called dew point (in the example 14°), i.e. a deposit in the form of mist (dew) is formed with further cooling. Hygrometers and psychrometers are used for the quantitative determination of humidity.

The relative equilibrium humidity of 85% at 23° C. can be adjusted to an accuracy of ±2% relative humidity (depending on the instrument used), for example in humidity-controlled laboratory chambers. Oversaturated solutions of certain salts also form constant and well-defined relative air humidities at a given temperature in closed systems, these relative air humidities being based on the phase equilibrium between the partial pressure of the water, the saturated solution and the sediment.

The combinations of shaped body and packaging system may of course themselves be packed in secondary packaging, for example in the form of cardboard boxes or trays, the secondary packaging having to meet the usual requirements. Accordingly, the secondary packaging is possible, but not necessary.

The packaging system surrounds one or more shaped bodies, depending on the embodiment of the invention. In one preferred embodiment of the invention, either a shaped body may be made up in such a way that it constitutes a dose or dosage unit of the colorant and may be individually packed or shaped bodies may be packed in a package in numbers which, together, constitute a dose or dosage unit. This principle may of course also be extended so that, according to the invention, combinations of three, four, five or even more shaped bodies may be accommodated in one and the same pack. Two or more shaped bodies in the same pack may of course have different compositions. In this way, certain components can be spatially separated from one another in order, for example, to avoid stability problems.

The packaging system of the combination according to the invention may consist of various materials and may assume various external forms. For economic reasons and in the interests of easier processability, however, preferred packaging systems are those in which the packaging material is light in weight, easy to process, inexpensive and ecologically safe.

In a first preferred combination according to the invention, the packaging system consists non-dimensionally stable packs, for example in the form of a bag of single-layer or laminated paper and/or plastic film. The shaped bodies may be introduced without sorting, i.e. loosely, into a bag of the materials mentioned above. However, for aesthetic reasons and for sorting the combinations in secondary packaging, bags are filled either with single tablets or with several shaped bodies in sorted form. These packaging systems may be optionally be packed—again preferably sorted—in outer packs which underscores the compact supply form of the shaped body.

The bags of single-layer or laminated paper or plastic film or metal foil preferably used as the packaging system may be designed in various ways, for example as inflated bags with no center seam or as bags with a center seam which are closed by heat (heat sealing), adhesives or adhesive tape. Single-layer bag materials are the known papers, which may optionally be impregnated, and plastic films which may optionally be co-extruded. Plastic films which may be used as the packaging system in accordance with the invention are described, for example, in Hans Domininghaus “Die Kunststoffe und ihre Eigenschaften” 3rd Edition, VDI Verlag, Düsseldorf, 1988, page 193. FIG. 111 of this publication also provides reference points in respect of the water vapor transmission of the materials mentioned.

Although wax-coated papers in the form of paperboard articles may also be used in addition to the films or papers mentioned as the packaging system for the shaped bodies, the packaging system preferably does not comprise any wax-coated paper.

In another embodiment, the shaped body is stored in dimensionally stable packaging, for example in the form of a blister. In this embodiment, the blister may be sealed with a metal foil or with corresponding film laminates.

The optional secondary packaging has to meet the usual requirements, so that any known materials and systems may be used.

In another embodiment, the packaging system is reclosable. It has proved to be practicable, for example, to use a reclosable tube of glass, plastic or even metal as the packaging system. The dosability of the hair coloring products can be optimized in this way, so that the consumer can be directed, for example, to use one tablet per defined unit of hair length. Packaging systems with a microperforation may also used with advantage for the purposes of the invention.

In a particularly preferred embodiment, the container K2 is attached to the packaging unit of the container K1. Thus, the container K2 may be mechanically joined, for example by coupling or fitting on, to the container K1. The two containers may also be adhesively joined to one another.

If the shaped body is accommodated in a blister, the blister is preferably attached to the packaging unit of the container K1 by making the seal of the blister act as a wall of the container K1. Accordingly, if the seal of the blister is broken by application of mechanical pressure to the blister or the shaped body, the shaped body has access to the medium M held in the container K1. This method of attachment enables the user—in the course of the process according to the invention—conveniently to dose the tablet into the medium M without coming into direct contact with it.

### EXAMPLES

The following shaped bodies for coloring hair were produced with a weight of 0.4 g and a fracture hardness of 60 to 80 N. The tablets were produced with a tabletting force of 3.5 kN.

Example 1

| | |
|---|---|
| 2-Methyl resorcinol | 19 mg |
| Resorcinol | 9 mg |
| Avicel ® pH 102¹ | 240 mg |
| Starlac ®² | 108 mg |
| Magnesium stearate | 4 mg |
| Colorona ® red-brown³ | 20 mg |

¹microcrystalline cellulose (FMC Corporation)
²mixture of lactose monohydrate and corn starch (ratio by weight 85:15) (Meggle)
³coated mica (INCI name: Mica, CI 77491 (Iron Oxides), CI 77891 (Titanium Dioxide)) (MERCK)

### Example 2

| | |
|---|---|
| 2,4-Diaminophenoxyethanol.2HCl | 25 mg |
| Avicel ® pH 102 | 240 mg |
| Starlac ® | 131 mg |
| Magnesium stearate | 4 mg |

### Example 3

| | |
|---|---|
| m-Aminophenol | 8 mg |
| 3-Amino-6-methoxy-2- | 2 mg |
| methylaminopyridine dihydrochloride | |
| Resorcinol | 31 mg |
| Avicel ® pH 102 | 240 mg |
| Starlac ® | 114 mg |
| Magnesium stearate | 4 mg |

1. A shaped body for coloring keratinous fibers comprising, in a cosmetically acceptable carrier, at least one dissolution accelerator and at least one oxidation dye precursor of the secondary intermediate type with the proviso that the shaped body is free from oxidation dye precursors of the primary intermediate type.

2. The shaped body of claim 1 wherein the dissolution accelerator is a disintegration aid.

3. The shaped body of claim 2 wherein the disintegration aid is cellulose-based.

4. The shaped body of claim 1 wherein the shaped body further comprises a mixture of starch and at least one saccharide.

5. The shaped body of claim 4 wherein the saccharide is a disaccharide.

6. The shaped body of claim 5 wherein the disaccharide is selected from the group consisting of lactose, maltose, sucrose, trehalose, turanose, gentobiose, melibiose and cellobiose.

7. The shaped body of claim 6 wherein the disaccharide is selected from the group consisting of lactose, maltose and sucrose.

8. The shaped body of claim 7 wherein the disaccharide is lactose.

9. The shaped body of claim 4 wherein the starch and the saccharides are present in a ratio by weight of 1:10 to 10:1.

10. The shaped body of claim 9 wherein the starch and the saccharides are present in a ratio by weight of 1:1 to 1:10.

11. The shaped body of claim 10 wherein the starch and the saccharides are present in a ratio by weight of 1:4 to 1:8.

12. The shaped body of claim 1 further comprising an alkalizing agent.

13. The shaped body of claim 1 further comprising at least one substantive dye.

14. The shaped body of claim 1 further comprising at least one pearlescent pigment.

15. The shaped body of claim 1 further comprising at least one bitter principle.

16. The shaped body of claim 1 wherein individual constituents of the composition to be compressed or the shaped body as a whole are coated.

17. The shaped body of claim 1 wherein the shaped body is enveloped in primary packaging.

18. The shaped body of claim 1 wherein the shaped body has a fracture hardness of 30 to 100 N (as measured to the Europäisches Arzneibuch 1997, 3rd Edition, ISBN 3-7692-2186-9, “2.9.8. Bruchfestigkeit von Tabletten (Fracture Resistance of Tablets)”; pages 143-144, using a Schleuniger 6D tablet hardness tester).

19. A method for coloring keratinous fibers comprising the steps of
(I) dissolving one or more of the shaped bodies of claim 1 in a medium M to form a preparation A,
(II) mixing preparation A with an oxidizing agent preparation B to form a ready-to-use colorant,
(III) applying the colorant F to the fibers and
(IV) rinsing the fibers with water after a contact time.

20. The method of claim 19 wherein the medium M has a viscosity of 500 to 100,000 mPa.s (Brookfield RVT viscosimeter, 4 r.p.m., spindle No. 4, temperature of 20° C.).

21. The method of claim 20 wherein the medium M is a gel or a w/o emulsion or o/w emulsion.

22. A kit for use with the method of claim 19 comprising three separate compartments K1, K2 and K3, wherein compartment K1 contains the medium M, compartment K2 contains the one or more shaped bodies of claim 1 and compartment K3 contains the oxidizing agent preparation B.
